SEQUENCE LISTING (14 sequences)

SEQ ID NO: 1 - DNA, 2341 bp, Artificial
  Fc gamma receptor
gattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtcctctccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccctatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaa

SEQ ID NO: 2 - protein, 290 aa, Artificial
  Fc gamma receptor
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 3 - DNA, 873 bp, Artificial
  Fc gamma receptor
atgggtggaggggctggggaaaggctgtttacttcctcctgtctagtcggtttggtccctttagggctccggatatctttggtgacttgtcctctccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 4 - protein, 290 aa, Artificial; identical to SEQ ID NO: 2
  Fc gamma receptor

SEQ ID NO: 5 - DNA, 765 bp, Artificial
  Fc gamma receptor
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 6 - protein, 254 aa, Artificial
  Fc gamma receptor
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 7 - DNA, 714 bp, Artificial
  Fc gamma receptor
atgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 8 - protein, 237 aa, Artificial
  Fc gamma receptor
MRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 9 - DNA, 765 bp, Artificial
  Fc gamma receptor
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 10 - protein, 254 aa, Artificial
  Fc gamma receptor
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 11 - DNA, 948 bp, Artificial
  Fc gamma receptor
atggagacccaaatgtctcagaatgtatgtcccagaaacctgtggctgcttcaaccattgacagttttgctgctgctggcttctgcagacagtcaagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccgtttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 12 - protein, 315 aa, Artificial
  Fc gamma receptor
METQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 13 - DNA, 402 bp, Artificial
  Fc gamma receptor
gcacctacttcaagttctgctaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcagagcatcatctcaacactgacttga

SEQ ID NO: 14 - protein, 133 aa, Artificial
  Fc gamma receptor
APTSSSAKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

(19) United States
(12) Patent Application Publication
(10) Pub. No.: US 2006/0165653 A1
(43) Pub. Date: Jul. 27, 2006

(54) Use of Fc receptor polymorphisms as diagnostics for treatment strategies for immune-response disorders

(75) Inventor: Susan E. Wilson, Alameda, CA (US)

Correspondence Address: Chiron Corporation;Intellectual Property - R440, P.O. Box 8097, Emeryville, CA, 94662-8097, US

(73) Assignee: Chiron Corporation, Emeryville, CA (US)

(21) Appl. No.: 11/305,843

(22) Filed: Dec. 16, 2005

Related U.S. Application Data
(63) Continuation of application No. 11/022,303, filed on Dec. 22, 2004, now abandoned.
(60) Provisional application No. 60/531,832, filed on Dec. 22, 2003.
(60) Provisional application No. 60/551,759, filed on Mar. 11, 2004.
(60) Provisional application No. 60/552,260, filed on Mar. 11, 2004.
(60) Provisional application No. 60/576,429, filed on Jun. 3, 2004.

(51) Int. Cl. A61K 38/20; C12Q 1/68; A61K 39/395

(52) U.S. Cl. 424/85.2; 424/145.1; 435/6

(57) ABSTRACT

Methods for the use of Fc gamma receptor (FcγR) polymorphisms as a diagnostic for intervention with interleukin-2 (IL-2) immunotherapy are provided. The methods comprise detecting the allelic pattern of an FcγRIIIA gene or FcγRIIA gene of an individual, and determining whether the allelic pattern is predictive of a positive therapeutic response to IL-2 immunotherapy. The presence of the FcγRIIIA 158F/F homozygous genotype, and/or the presence of one or both copies of the FcγRIIIA 48L allele, and/or the presence of one or both copies of the FcγRIIA 131R allele is predictive of a positive therapeutic response to IL-2 immunotherapy, and therefore indicative of medical intervention with IL-2 immunotherapy for treatment of an immune disorder. The diagnostic method finds use in identifying those individuals whose immune function can be improved by treatment with IL-2 immunotherapy, particularly for individuals with cancer.

### FIELD OF THE INVENTION

The present invention is directed to the field of predictive medicine, more particularly the use of Fc gamma receptor (FcγR) polymorphisms as diagnostics for assessing treatment strategies in immune-response disorders.

### BACKGROUND OF THE INVENTION

Interleukin-2 (IL-2) is a potent stimulator of natural killer (NK) and T-cell proliferation and function (Morgan et al. (1976) Science 193:1007-1011). This naturally occurring lymphokine has been shown to have anti-tumor activity against a variety of malignancies either alone or when combined with lymphokine-activated killer (LAK) cells or tumor-infiltrating lymphocytes (TIL) (see, for example, Rosenberg et al. (1987) N. Engl. J. Med. 316:889-897; Rosenberg (1988) Ann. Surg. 208:121-135; Topalian et al. 1988) J. Clin. Oncol. 6:839-853; Rosenberg et al. (1988) N. Engl. J. Med. 319:1676-1680; and Weber et al. (1992) J. Clin. Oncol. 10:33-40). The anti-tumor activity of IL-2 has best been described in patients with metastatic melanoma and renal cell carcinoma using Proleukin®, a commercially available IL-2 formulation. Other diseases, including lymphoma, also appear to respond to treatment with IL-2 (Gisselbrecht et al. (1994) Blood 83(8):2020-2022). However, high doses of IL-2 used to achieve positive therapeutic results with respect to tumor growth frequently cause severe side effects, including fever and chills, hypotension and capillary leak (vascular leak syndrome or VLS), and neurological changes (see, for example, Duggan et al. (1992) J. Immunotherapy 12:115-122; Gisselbrecht et al. (1994) Blood 83:2081-2085; and Sznol and Parkinson 1994) Blood 83:2020-2022).

Monoclonal antibodies have increasingly become a method of choice for the treatment of solid tumors, for example breast cancer, as well as for treatment of lymphomas of the B-cell type, which express the CD20 cell surface antigen. In vitro work has demonstrated that monoclonal antibodies directed to CD20 result in cell death by apoptosis (Shan et al. (1998) Blood 91:1644-1652). Other studies report that B-cell death is primarily mediated by antibody-dependent cytotoxicity (ADCC).

Because of the possible immunological basis of the anti-tumor activity of anti-CD20 antibodies, combinations with other cytokines that enhance NK cell function have been examined. Cytokines such as IL-12, IL-15, TNF-alpha, TNF-beta, gamma-IFN, and IL-2 have been tested for potentiation of ADCC, a distinct NK function. All appear to be active in enhancing ADCC, although each agent is associated with its own specific toxicities. See, e.g., Rosenberg et al. (1986) Science 233(4770):1318-1321; Gollob et al. (1998) J Clin Invest. 102(3):561-575. Ongoing studies of combination therapy with IL-2 and the monoclonal antibody rituximab (Rituxan®; IDEC-C2B8; IDEC Pharmaceuticals Corp., San Diego, Calif.) have shown improved clinical response in non-Hodgkin's B-cell lymphoma patients (U.S. Patent Publication 20030185796) with these two therapeutic agents.

Rituximab is a chimeric anti-CD20 monoclonal antibody containing human IgG1 and kappa constant regions with murine variable regions isolated from a murine anti-CD20 monoclonal antibody, IDEC-2B8 (Reff et al. (1994) Blood 83:435-445). Rituximab has been shown to be an effective treatment for low-intermediate and high-grade non-Hodgkin's lymphoma (see, for example, Maloney et al. (1994) Blood 84:2457-2466); McLaughlin et al. (1998) J. Clin. Oncol. 16:2825-2833; Maloney et al. (1997) Blood 90:2188-2195; Hainsworth et. al. (2000) Blood 95:3052-3056; Colombat et al. (2001) Blood 97:101-106; Coiffier et al. (1998) Blood 92:1927-1932); Foran et al. (2000) J. Clin. Oncol. 18:317-324; Anderson et al. (1997) Biochem. Soc. Trans. 25:705-708; Vose et al. (1999) Ann. Oncol. 10:58a). However, 30% to 50% of patients with low-grade NHL exhibit no clinical response to this monoclonal antibody (Hainsworth et. al. (2000) Blood 95:3052-3056; Colombat et al. (2001) Blood 97:101-106). Though the exact mechanism of action is not known, evidence indicates that the anti-lymphoma effects of rituximab are in part due to complement mediated cytotoxicity (CMC), antibody-dependent cell mediated cytotoxicity (ADCC), inhibition of cell proliferation, and finally direct induction of apoptosis.

ADCC is mediated through leukocyte receptors for the Fc portion of IgG (FcγR). The Fc receptors are membrane bound glycoproteins that are expressed on the surface of neutrophils, macrophages, and other cell types whose primary function is to bind and internalize immunoglobulins, immune complexes, and other particles. Different types of FcγR may be expressed on various immune effector cells. Engagement of specific FcγRs results in activation or inhibition of the effector cell.

The FcγRs identified thus far have been assigned to three classes: FcγRI(CD64), FcγRIIA (CD32), and FcγRIIIA (CD16) activate effector cells; FcγRIIB inhibits activation; and FcγRIIIB cooperates with other FcγRs. FcγRIIIA is located on NK cells, macrophages, and monocytes, while FcγRIIA and FcγRIIB are predominately expressed on macrophages and not on NK cells. Engagement of activating receptors promotes immune activity, such as cytokine release and inflammatory reactions, while engagement of inhibitory receptors primarily results in clearance of immune complexes without immune activation.

In ADCC, rituximab binds to CD20 antigen on the surface of cancer cells, and then bridges the effector cells, such as NK cells and macrophages, via the FcγR on these effector cells. Natural killer cells, which account for approximately 15% of human peripheral blood lymphocytes, are the principle effector cells that mediate ADCC against tumor cells. The low affinity FcγRIIIA receptor on the surface of NK cells recognizes and binds to IgG antibodies. Engagement of FcγRIIIA on NK cells is considered to be a fundamental mechanism contributing to the anti-tumor activity of therapeutically administered IgG monoclonal antibodies such as rituximab (Clynes et al. (2000) Nature Med. 6:443-446; Cooper et al. (2001) Trends Immunol. 22:633-640; Leibson (1997) Immunity 6:655-661; Roitt et al. (2001) Immunology (6th ed.; Mosby, Edinburgh, UK). NK cell cytotoxicity is activated by cytokines such as IL-2 and IL-12.

Recently three polymorphisms of these FcγRs having different binding affinities for specific IgG subclasses have been identified: a polymorphism of FcγRIIIA at position 158 of the mature sequence with either a valine (V) or phenylalanine (F) residue, a triallelic polymorphism of FcγRIIIA at position 48 of the mature sequence with either a leucine (L), arginine (R), or histidine (H) residue, and a polymorphism of FcγRIIA at position 131 of the mature sequence with either a histidine (H) or arginine (R) residue. The FcγRIIIA 158V allele binds human IgG1 better than does the FcγRIIIA 158F allele (Koene et al. (1997) Blood 90:1109-1114), and the increased binding of the 158V allele results in enhanced activation of effector cells and better ADCC (Shields et al. (2001) J. Biol. Chem. 176:6591-6604; Vance et al. (1993) J. Immunol. 151:6429-6439). The FcγRIIA 48R and FcγRIIIA 48H alleles reportedly have a higher affinity for human IgG1, IgG3, and IgG4 than does the FcγRIIIA 48L allele (de Haas et al. (1996) J. Immunol. 156(8):3948-3955). The FcγRIIA 131H allele has higher affinity for IgG2 than does the FcγRIIA 131R allele, though no significant difference in the affinity of these allelic forms for IgG1 has been reported (Parren et al. (1992) J. Clin. Invest. 90:1537-1546). As a consequence, homozygosity for 48L/L of FcγRIIIA, 158F/F of FcγRIIIA, or 131R/R of FcγRIIA lessens the ability to interact with specific IgG subclasses. The latter two of these polymorphisms have been found to be predictors of clinical response to rituximab. Thus, a higher rituximab response rate is associated with the FcγRIIIA 158V/V genotype (Cartron et al. (2002) Blood 99:754-758; Weng and Levy (2003) J. Clin. Oncol. 21:1-8) or the FcγRIIA 13 IH/H genotype (Weng and Levy (2003) J. Clin. Oncol. 21:1-8). Furthermore, those individuals having both the FcγRIIIA 158V/V and the FcγRIIA 131H/H genotypes had long-lasting remissions (Weng and Levy (2003) J. Clin. Oncol. 21:1-8).

Given the importance of these polymorphisms in responsiveness to monoclonal antibody therapy, other means by which these polymorphisms can be used as diagnostics for clinical response to other immune modulators are needed.

### SUMMARY OF THE INVENTION

Methods for the use of Fc gamma receptor (FcγR) polymorphisms as a diagnostic for intervention with interleukin-2 (IL-2) immunotherapy are provided. The methods comprise detecting the allelic pattern of an FcγRIIIA gene or FcγRIIA gene of an individual, and determining whether the allelic pattern is predictive of a positive therapeutic response to IL-2 immunotherapy. The presence of the FcγRIIIA 158F/F homozygous genotype, and/or the presence of one or both copies of the FcγRIIIA 48L allele, and/or the presence of one or both copies of the FcγRIIA 131R allele is predictive of a positive therapeutic response to IL-2 immunotherapy, and therefore indicative of medical intervention with IL-2 immunotherapy for treatment of an immune disorder.

The methods find use in identifying those individuals whose immune response is compromised, and for which IL-2 immunotherapy can provide a means for enhancing their ability to effectively mount an FcγR-mediated immune response. Thus, the present invention also provides methods for treating an immune disorder in individuals carrying these particular FcγR polymorphisms, where treatment comprises administering IL-2 immunotherapy, alone or in combination with one or more other agents that provide a therapeutic effect via an FcγRIIIA-mediated or FcγRIIA-mediated immune response. Immune disorders that can be treated using the methods of the present invention include, but are not limited to, cancers such as the B-cell lymphomas and solid tumors, including breast, colon, ovarian, cervical, prostate, and other cancers.

In one aspect, the invention includes a diagnostic method for predicting therapeutic response to interleukin-2 (IL-2) immunotherapy in an individual in need thereof, the method comprising detecting the allelic pattern for the Fc gamma receptor IIIA (FcγRIIIA) gene of the individual, wherein the presence of the homozygous FcγRIIIA 158F/F genotype is indicative of an individual that will exhibit a positive therapeutic response to the IL-2 immunotherapy.

In another aspect, the invention includes a diagnostic method for predicting therapeutic response to interleukin-2 (IL-2) immunotherapy in an individual in need thereof, the method comprising detecting the allelic pattern for the Fc gamma receptor IIA (FcγRIIA) gene of the individual, wherein the presence of the heterozygous FcγRIIA 131H/R genotype or the presence of the homozygous FcγRIIA 131R/R genotype is indicative of an individual that will exhibit a positive therapeutic response to the IL-2 immunotherapy.

In yet another aspect, described herein is a method for enhancing immune function of an individual that comprises the homozygous Fc gamma RIIIA (FcγRIIIA) 158F/F genotype, the method comprising administering interleukin-2 immunotherapy to the individual.

In yet another aspect, the invention includes a method for enhancing immune function of an individual that comprises the heterozygous Fc gamma receptor IIA (FcγRIIA) 131H/R genotype or the homozygous FcγRIIA 131R/R genotype, the method comprising administering interleukin-2 immunotherapy to the individual.

In a still further aspect, the invention includes a method for treating a cancer in an individual comprising a homozygous Fc gamma IIIA (FcγRIIIA) 158F/F genotype, the method comprising administering interleukin-2 immunotherapy to the individual.

In another aspect, the invention comprises a method for treating a cancer in an individual comprising a heterozygous Fc gamma IIA (FcγRIIA) 131H/R genotype or a homozygous FcγRIIA 131R/R genotype, the method comprising administering interleukin-2 immunotherapy to the individual.

In yet another aspect, the invention provides a diagnostic method for predicting therapeutic response to interleukin-2 (IL-2) immunotherapy in an individual in need thereof, the method comprising detecting the allelic pattern for the Fc gamma receptor IIIA (FcγRIIIA) gene of the individual, wherein the presence of the homozygous FcγRIIIA 48L/L genotype, the heterozygous FcγRIIIA 48L/R genotype, or the heterozygous FcγRIIIA 48L/H genotype is indicative of an individual that will exhibit a positive therapeutic response to the IL-2 immunotherapy. In certain embodiments, the allelic pattern for the FcγRIIIA gene is detected by a method selected from the group consisting of allele specific hybridization, primer specific extension, oligonucleotides ligation assay, restriction enzyme site analysis, and single-stranded conformation polymorphism analysis.

In yet another aspect, the invention includes a method for enhancing immune function of an individual that comprises the homozygous Fc gamma RIIIA (FcγRIIIA) 48L/L genotype, the method comprising administering interleukin-2 immunotherapy to the individual.

In a still further aspect, described herein is a method for treating a cancer in an individual comprising a heterozygous Fc gamma IIA (FcγRIIA) 131H/R genotype or a homozygous FcγRIIA 131R/R genotype, the method comprising administering interleukin-2 immunotherapy to the individual.

In any of the methods described herein, the individual may be in need of, or may be undergoing, treatment of a cancer, for example IL-2 immunotherapy and/or treatment with an antibody that targets a cell-surface antigen expressed on the surface of cells of the cancer, for example an immunoglobulin G1 (IgG1) monoclonal antibody. The cancer may be a B-cell lymphoma (e.g., non-Hodgkin's B-cell lymphoma), breast cancer, ovarian cancer, cervical cancer, prostate cancer, colon cancer, melanoma, renal cell carcinoma, acute myeloid leukemia (AML) or chronic lymphocytic leukemia (CLL).

In any of the methods described herein, the IL-2 immunotherapy may comprise administering at least one therapeutically effective dose of IL-2 or biologically active variant thereof to the individual. Alternatively, in any of the methods described herein, multiple therapeutically effective doses of IL-2 or variant thereof may be administered to the individual, for example, according to a daily dosing regimen, a twice-a-week or three-times-a-week dosing regimen.

In any of the methods described herein, the IL-2 or variant thereof may be administered subcutaneously. Furthermore, in any of these methods, the IL-2 or variant thereof may be provided in a pharmaceutical composition selected from the group consisting of a monomeric IL-2 pharmaceutical composition, a multimeric IL-2 composition, a lyophilized IL-2 pharmaceutical composition, and a spray-dried IL-2 pharmaceutical composition. Furthermore, the IL-2 may be recombinantly produced IL-2 having an amino acid sequence for human IL-2 or a variant thereof having at least 70% sequence identity to the amino acid sequence for human IL-2, for example, des-alanyl-1, serine 125 human interleukin-2.

Any of the methods described herein may further comprise administering to the individual an immunoglobulin G1 (IgG1) monoclonal antibody.

Furthermore, in any of the methods described herein, the individual may also be being treated for a cancer (e.g., a B-cell lymphoma such as non-Hodgkin's B-cell lymphoma, breast cancer, ovarian cancer, cervical cancer, prostate cancer, colon cancer, melanoma, renal cell carcinoma, acute myeloid leukemia (AML), and chronic lymphocytic leukemia (CLL)). The cancer treatment may comprise an IgG1 monoclonal antibody that is an anti-CD20 antibody or antigen-binding fragment thereof, for example Therex, MDX-010, EMD 72000, Erbitux, WX-G250, IDM-1, MDX-210, ZAMYL, Campath, and antigen-binding fragments thereof.

In addition, in any of them methods described herein, the allelic pattern for the FcγRIIIA gene may be detected by a method selected from the group consisting of allele specific hybridization, primer specific extension, oligonucleotides ligation assay, restriction enzyme site analysis, and single-stranded conformation polymorphism analysis.

In another aspect, the invention includes a kit for use in a diagnostic method for predicting therapeutic response to interleukin-2 (IL-2) immunotherapy in an individual in need thereof, the kit comprising at least one probe or primer that specifically hybridizes adjacent to or at a polymorphic region of the Fc gamma receptor IIIA (FcγRIIA) gene, the polymorphic region comprising nucleotides encoding the FcγRIIIA 158F allele.

In a still further aspect, the invention comprises a kit for use in a diagnostic method for predicting therapeutic response to interleukin-2 (IL-2) immunotherapy in an individual in need thereof, the kit comprising at least one probe or primer that specifically hybridizes adjacent to or at a polymorphic region of the Fc gamma receptor IIA (FcγRIIA) gene, the polymorphic region comprising nucleotides encoding the FcγRIIA 131R allele.

In another aspect, provided herein is a kit for use in a diagnostic method for predicting therapeutic response to interleukin-2 (IL-2) immunotherapy in an individual in need thereof, the kit comprising at least one probe or primer that specifically hybridizes adjacent to or at a polymorphic region of the Fc gamma receptor IIIA (FcγRIIIA) gene, the polymorphic region comprising nucleotides encoding the FcγRIIIA 48L allele.

Any of the kits described herein may further comprise instructions for use.

### BRIEF DESCRIPTION OF THE DRAWINGS

FIG. 1 diagrams the location of the FcγRIIIA 158 V/F polymorphism, which is dependent upon which of the three possible start codons within SEQ ID NO:1 are used to initiate the open reading frame for the human FcγRIIIA sequence. Where translation begins at nucleotide 185 of SEQ ID NO:1 (i.e., the first start codon), the G/T substitution results in the V/F polymorphism occurring at amino acid residue 212 of the translated polypeptide (see SEQ ID NO:4). Where translation begins at nucleotide 293 of SEQ ID NO:1 (i.e., the second start codon), the G/T substitution results in the V/F polymorphism occurring at amino acid residue 176 of the translated polypeptide (see SEQ ID NO:6). Where translation begins at nucleotide 344 of SEQ ID NO:1 (i.e., the third start codon), the G/T substitution results in the V/F polymorphism occurring at amino acid residue 159 of the translated polypeptide (see SEQ ID NO:8).

FIG. 2 shows the correlation of CD16/56+ NK cell count and clinical status for the FcγRIIIA 158 F/F patient subset in the IL2NHL03 study described in the Experimental section herein below.

FIG. 3 is a graph depicting the percent change in tumor volume in genotyped patients, measured eight weeks after starting combination rituximab-IL-2 administration. The administration regime is described in detail in Example 4.

FIG. 4, panels A and B, depict alignments of nucleotide sequences from FcγRIIIa and FcγRIIIb genes. FIG. 4A aligns partial cDNA sequence from FCγRIIIa (top line, labeled HSFCGR31 and also referred to as gene B) and FcγRIIIb (bottom line, labeled HSFCGR32 and also referred to as gene A). Also shown in FIG. 4A in boxes are: positions indicating gene A or gene B (position 473, 531 and 641) as well as the single nucleotide polymorphism (occurring only in gene A) at position 559 that predicts a V→F substitution. FIG. 4B aligns exon 4 of gene A and gene B and shows various nucleotide differences between the two genes, including the highly specific nucleotide variation at position 313, numbered relative to the first base of exon 4.

FIG. 5, panels A through N, depict SEQ ID NOs:1 through 14.

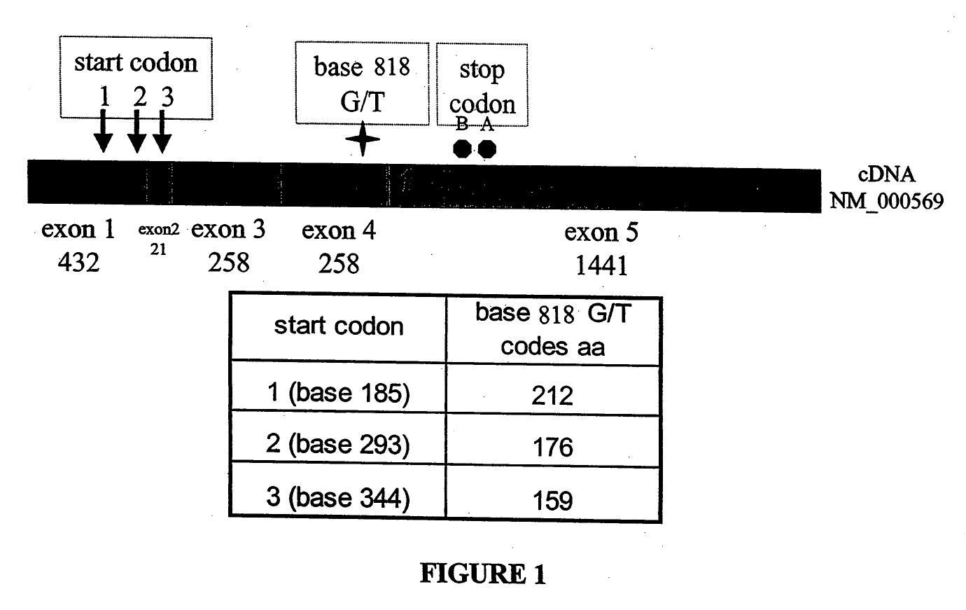

### DETAILED DESCRIPTION OF THE INVENTION

The present invention relates to diagnostic methods for predicting therapeutic response to interleukin-2 (IL-2) immunotherapy in a human subject in need thereof, particularly individuals that are contemplating IL-2 immunotherapy in combination with an anti-cancer monoclonal antibody that mediates its therapeutic effect via receptor-mediated antibody-dependent cellular cytotoxicity (ADCC). The methods of the invention utilize Fc gamma receptor (FcγR) functional polymorphisms as a diagnostic tool to determine whether intervention with IL-2 immunotherapy is likely to provide a positive therapeutic response. Of particular interest are the valine (V)/phenylalanine (F) polymorphism at position 158 of mature human FcγRIIIA (corresponding to position 176 of SEQ ID NO:6, where the V residue is shown; encoded by the nucleotide sequence shown in SEQ ID NO:5; the leucine (L)/arginine (R)/histidine (H) triallelic polymorphism at position 48 of mature human FcγRIIIA (corresponding to position 66 of SEQ ID NO:6, where the L residue is shown; encoded by the nucleotide sequence shown in SEQ ID NO:5); and the histidine (H)/arginine (R) polymorphism at position 131 of mature human FcγRIIA (corresponding to position 165 of SEQ ID NO:12, where the R residue is shown; encoded by the nucleotide sequence shown in SEQ ID NO:11 (GenBank Accession No. NM_{—}021642)).

The FcγRIIIA 158 V/F polymorphism has been referred to in the scientific literature as both the 158 V/F polymorphism and the 176 V/F polymorphism, depending upon whether the mature FcγRIIIA sequence or precursor FcγRIIIA sequence serves as the reference for numbering the location of this polymorphism. For purposes of the present invention, these two terms are used interchangeably. The full-length sequence encoding human FcγRIIIA is set forth in SEQ ID NO:1, with the translated amino acid sequence set forth in SEQ ID NO:2. See GenBank Accession No. NM_{—}000569. This coding sequence comprises 3 possible translation initiation codons. Where translation begins at nucleotide 185 of SEQ ID NO:1 (i.e., the first start codon), the G/T substitution results in the V/F polymorphism occurring at amino acid residue 212 of the translated polypeptide (see SEQ ID NO:4, encoded by SEQ ID NO:3). Where translation begins at nucleotide 293 of SEQ ID NO:1 (i.e., the second start codon), the G/T substitution results in the V/F polymorphism occurring at amino acid residue 176 of the translated polypeptide (see SEQ ID NO:6, encoded by SEQ ID NO:5). Where translation begins at nucleotide 344 of SEQ ID NO:1 (i.e., the third start codon), the G/T substitution results in the V/F polymorphism occurring at amino acid residue 159 of the translated polypeptide (see SEQ ID NO:8, encoded by SEQ ID NO:7). All of these sequences show the V residue at the respective location of the polymorphism. The exact position of the G/T substitution that results in the substitution of a phenylalanine (F) residue for the valine (V) residue resides at nucleotide 818 of SEQ ID NO:1, nucleotide 634 of SEQ ID NO:3, nucleotide 526 of SEQ ID NO:5, and nucleotide 475 of SEQ ID NO:7. For purposes of the present invention, the second translation initiation codon serves as the initiation site, and hence the translated polypeptide has the sequence set forth in SEQ ID NO:6, which is encoded by SEQ ID NO:5. The G/T substitution at position 526 of SEQ ID NO:5 results in the sequence shown in SEQ ID NO:9, which encodes the human FcγRIIIA polypeptide of SEQ ID NO:10 showing the phenylalanine (F) residue at position 176 of this sequence. This corresponds to a phenylalanine substitution at position 158 of the mature human FcγRIIIA sequence. The polymorphism at position 158 of mature human FcγRIIIA results in three possible genotypes. An individual who has two copies of the 158V allele is designated as having the homozygous FcγRIIIA 158V/V genotype, while an individual who has two copies of the 158F allele is designated as having the homozygous FcγRIIIA 158F/F genotype. Individuals having a copy of both the 158V and 158F alleles are designated as having the heterozygous FcγRIIIA 158V/F genotype.

The FcγRIIIA 48 L/R/H triallelic polymorphism has been referred to in the scientific literature as both the FcγRIIIA 48 L/R/H polymorphism and the FcγRIIIA 66 L/R/H polymorphism, depending upon whether the mature FcγRIIIA sequence or precursor FcγRIIIA sequence, respectively, serves as the reference for numbering the location of this polymorphism. For purposes of the present invention, these two terms are used interchangeably. Where translation begins at nucleotide 185 of SEQ ID NO:1 (i.e., the first start codon), the T/G substitution or the T/A substitution results in the L/R or L/H polymorphism, respectively, occurring at amino acid residue 102 of the translated polypeptide (see SEQ ID NO:4, encoded by SEQ ID NO:3). Where translation begins at nucleotide 293 of SEQ ID NO:1 (i.e., the second start codon), the T/G substitution or the T/A substitution results in the L/R or L/H polymorphism, respectively, occurring at amino acid residue 66 of the translated polypeptide (see SEQ ID NO:6, encoded by SEQ ID NO:5). Where translation begins at nucleotide 344 of SEQ ID NO:1 (i.e., the third start codon), the T/G substitution or the T/A substitution results in the LIR or L/H polymorphism, respectively, occurring at amino acid residue 49 of the translated polypeptide (see SEQ ID NO:8, encoded by SEQ ID NO:7). All of these sequences show the L residue at the respective location of the polymorphism. The exact position of the T/G substitution or the T/A substitution that results in the substitution of an arginine (R) or histidine (H) residue for the leucine (L) residue resides at nucleotide 489 of SEQ ID NO:1, nucleotide 305 of SEQ ID NO:3, nucleotide 197 of SEQ ID NO:5, and nucleotide 146 of SEQ ID NO:7. For purposes of the present invention, the second translation initiation codon serves as the initiation site, and hence the translated polypeptide has the sequence set forth in SEQ ID NO:6, which is encoded by SEQ ID NO:5. The T/G substitution or the T/A substitution at position 197 of SEQ ID NO:5 results in a substitution of an arginine (R) or histidine (H) for the leucine (L) at position 66 of SEQ ID NO:6. This corresponds to an arginine (R) or histidine (H) substitution for the leucine (L) at position 48 of the mature human FcγRIIIA sequence. The triallelic polymorphism at position 48 of mature human FcγRIIIA results in the following possible L-carrying genotypes of interest to the present invention. An individual who has two copies of the 48L allele is designated as having the homozygous FcγRIIIA 48 L/L genotype. Individuals having a copy of both the 48L and 48R alleles are designated as having the heterozygous FcγRIIIA 48L/R genotype, while individuals having a copy of both the 48L and 48H alleles are designated as having the heterozygous FcγRIIIA 48 L/H genotype.

The “conventional” version of the DNA encoding FcγRIIA contains a G (guanine) at position 494 of SEQ ID NO:11; while the “polymorphic” version contains an A (adenine) at this position. The substitution of A for G results in a change in the amino acid residue encoded at position 165 of SEQ ID NO:12 from arginine to histidine, which corresponds to position 131 of the mature human FcγRIIA sequence. The polymorphism at position 131 of mature human FcγRIIA results in the following three genotypes: homozygous FcγRIIA 131H/H, homogygous FcγRIIA 131R/R, and heterozygous FcγRIIA 131H/R.

Individuals carrying one or more copies of the low affinity FcγRIIIA 158F allele and/or one or more copies of the low affinity FcγRIIIA 48L allele, and/or one or more copies of the low affinity FcγRIIA 131R allele have a defective FcγR-mediated immune response compared to individuals carrying both copies of the high affinity FcγRIIIA 158V allele, and/or both copies of the FcγRIIIA 48H or 48R allele, and/or both copies of the high affinity FcγRIIA 13 1H allele. By “FcγR-mediated immune response” is intended an immune response, particularly mediated via ADCC, that results in a lessening or amelioration of at least one symptom of the immune disorder for which the individual is undergoing treatment. By “defective” is intended the individual, when presented with an agent that mediates its cytotoxic effect via its interaction with an FcγR, is unable to mount an effective FcγR-mediated immune response, and thus presentation of the agent fails to elicit a positive therapeutic response. Such individuals are resistant to anti-cancer monoclonal antibodies that mediate their cytotoxity via IgG interaction with activating FcγRs, particularly via FcγRIIIA or FcγRIIA.

The present invention is based on the discovery that intervention with interleukin-2 (IL-2) immunotherapy can convert individuals carrying the homozygous FcγRIIIA 158F/F genotype and/or the heterozygous FcγRIIA 131H/R or homozygous FcγRIIA 131R/R genotype to a responsive state. By “responsive state” is intended the individual, when presented with an agent that mediates its cytotoxic effect via its interaction with an FcγR, is able to mount an effective FcγR-mediated immune response, and thus presentation of the agent elicits a positive therapeutic response.

Without being bound by theory, intervention with IL-2 immunotherapy can induce expansion and activation of FcγR-bearing cells including natural killer (NK) cells, monocytes/macrophages, and neutrophils, thereby augmenting the ADCC-mediated cytotoxic effects of a therapeutic agent, for example, an anti-cancer antibody. As a result, immunotherapeutic intervention with IL-2 or biologically active variant thereof may achieve a critical threshold sufficient to drive ADCC more effectively in individuals carrying low affinity IgG FcγRIIIA and/or FcγRIIA allotypes.

Furthermore, and again without being bound by theory, the overall response to IL-2 immunotherapy in combination with anti-cancer therapeutic agents that depend on ADCC-mediated cytotoxicity via interaction with FcγR for their therapeutic effect, such as an anti-cancer antibody, appears to be dependent upon three key variables: level of expression of the tumor antigen, expansion of NK cell number following administration of IL-2, and FcγR genotype. Thus, for example, where a subject is going to undergo cancer treatment with rituximab (Rituxan®; IDEC Pharmaceuticals Corp., San Diego, Calif.), initial therapeutic response is going to be dependent upon level of expression of the CD20 antigen on the tumor being treated. Certain NHL histologies, for example, chronic lymphocytic leukemia, plasmacytoid, express low level CD20 antigen levels and are therefore less likely to respond to CD20 targeted therapeutics, e.g., rituximab. In addition, repeated use of rituximab can drive a tumor escape mechanism whereby tumor CD20 expression is downregulated. IL-2 expansion of NK cells predictably would be less effective in restoring rituximab responses in individuals with low/absent tumor CD20 antigen expression. By way of another example, individuals that are carriers for the FcγRIIIA 158V allele (i.e., FcγRIIIA 158V/V or 158 V/F genotype) should respond to rituximab alone; where response rate is low, it could be related to low-level expression of CD210 as a consequence of poor responder histology (e.g., CLL and plamacytoid) or tumor evasion in response to prior repeated rituximab usage.

Again, without being bound by theory, expansion of NK cell number following IL-2 treatment (i.e., IL-2-induced immune reconstitution) may be key to determining the overall response to rituximab/IL-2 combination therapy in rituximab relapsed/refractory subjects. Low NK cell numbers result in inefficient ADCC. NK expansion following IL-2 administration above a theoretical critical threshold serves to restore/drive efficient rituximab usage.

Finally, and again without being bound by theory, FcγR genotype plays a role in overall response rate. The FcγRIIIA 158V allele binds with highest affinity to IgG1 and therefore overall clinical response rates to rituximab IgG1 antibody is predictably highest in FcγRIIIA 158V/V carriers. However, IL-2 may also restore efficient FcR cell-mediated ADCC in individuals who have FcγRIIIA 158V/V or 158V/F phenotypes but have impaired or damaged immune systems as a result of chemotherapy/radiotherapy or as a consequence of age. The FcγRIIIA 158 polymorphism appears to be predominant in determining affinity for IgG1 and there is clear but in complete linkage with the triallelic L/R/H polymorphism at position 48 of mature human FcγRIIIA. The FcγRIIIA 158F allele shows lower binding affinity for IgG1 and therefore IL-2 more likely offers the most benefit in augmenting ADCC in FcγRIIIA 158 F/F carriers. Similarly, FcγRIIIA 48L binds with lower affinity to IgG1 than either the 48R or 48H alleles, and therefore it is predicted that IL-2 will offer most benefit to FcγRIIIA 48L carriers, i.e., FcγRIIIA 48 L/L, FcγRIIIA 48 L/R, or FcγRIIIA 48L/H genotypes.

By “IL-2 immunotherapy” is intended administration of at least one therapeutically effective dose of IL-2 or biologically active variant thereof as defined herein below. By “therapeutically effective dose or amount” of IL-2 or variant thereof is intended an amount of the IL-2 or variant thereof that, when administered, brings about a positive therapeutic response with respect to treatment of an individual for an immune response, particularly a cancer. Of particular interest is an amount of IL-2 or variant thereof that converts an individual who carries the homozygous FcγRIIIA 158F/F genotype and/or the heterozygous FcγRIIA 131 HIR or homozygous FcγRIIA 131 R/R genotype to a responsive state as noted above.

Where IL-2 immunotherapy contemplates administration of multiple therapeutically effective doses, the IL-2 or variant thereof can be administered according to a daily dosing regimen, or can be administered intermittently. By “intermittent” administration of IL-2 or variant thereof is intended the therapeutically effective dose can be administered, for example, every other day, every two days, every three days, and so forth. In some embodiments, IL-2 immunotherapy comprises twice-weekly administration or thrice-weekly administration of a therapeutically effective dose of IL-2 or variant thereof By “twice-weekly” or “two times per week” is intended two therapeutically effective doses of IL-2 or variant thereof are administered to the subject within a 7 day period, beginning on day 1 of the first week of IL-2 administration, with a minimum of 72 hours between doses and a maximum of 96 hours between doses. By “thrice weekly” or “three times per week” is intended three therapeutically effective doses of IL-2 or variant thereof are administered to the subject within a 7 day period, allowing for a minimum of 48 hours between doses and a maximum of 72 hours between doses. For purposes of the present invention, this type of IL-2 dosing is referred to as “intermittent IL-2 immunotherapy.” In accordance with the methods of the present invention, a subject can receive intermittent IL-2 immunotherapy with IL-2 or variant thereof (i.e., twice-weekly or thrice-weekly administration of a therapeutically effective dose of IL-2 or variant thereof) for one or more weekly cycles until the desired therapeutic response is achieved. The IL-2 or variant thereof can be administered by any acceptable route of administration as noted herein below.

Thus, the present invention provides a diagnostic method for predicting therapeutic response to IL-2 immunotherapy in an individual in need thereof, particularly an individual that is undergoing therapy with a second agent that mediates its cytotoxic effect via its interaction with an FcγR. The methods comprise detecting the allelic pattern for the FcγRIIIA gene, and/or the FcγRIIA gene, of an individual, and thereby ascertaining the individual's genotype for that FcγR gene. The presence of the homozygous FcγRIIIA 158F/F genotype, and/or the presence of at least one copy of the FcγRIIA 131R allele, is indicative of an individual for whom intervention with IL-2 immunotherapy will provide a positive therapeutic response. By “positive therapeutic response” is intended the individual undergoing IL-2 immunotherapy exhibits an improvement in one or more symptoms of the immune disorder for which the individual is undergoing therapy.

Thus, for example, where the individual is suffering from a cancer, including those cancers identified herein below, a “positive therapeutic response” would be an improvement in the disease in association with IL-2 immunotherapy, and/or an improvement in one or more symptoms of the disease in association with IL-2 immunotherapy. The IL-2 immunotherapy could be the sole line of treatment to which the individual is exposed. Alternatively, the IL-2 immunotherapy could be administered concurrently with a second therapeutic agent, particularly an anti-cancer agent that mediates its cytotoxic effects via its interaction with FcγRIIIA and/or FcγRIIA. Thus, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, nuclear and radiographic studies (i.e., CT (computer tomography) and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements (for patients with evaluable response only, partial response does not apply).

In one embodiment, the agent being administered in combination with IL-2 immunotherapy is an anti-cancer antibody, particularly monoclonal antibodies that mediate their cytotoxicity effects via IgG1/FcγR-mediated ADCC. Such monoclonal antibodies include, but are not limited to, Rituxan® (which targets the CD20 antigen on neoplastic B cells, and is effective for treatment of B-cell lymphomas, including non-Hodgkin's B-cell lymphomas, and chronic lymphocytic leukemia (CLL)); Therex (humanized HMFG1 specific for MUC1, which is being developed for breast cancer) and other MUC1-positive tumors including ovarian and colon cancers); MDX-010 (human anti-CTLA-4 negative regulator on activated T cells; being developed for melanoma, follicular lymphoma, colon, and prostate cancers); EMD 72000 and Erbitux (IMC-225) (human anti-EGFR being developed for EGFR-positive cancers, most notably colon carcinoma); WX-G250 (specific for MN antigen; being developed for renal cell carcinoma and cervical cancer); IDM-1 (for treatment of ovarian cancer); MDX-210 (for treatment of breast and ovarian cancer); ZAMYL (for treatment of acute myeloid leukemia (AML)); and Campath (for treatment of CLL). The individual is administered one or more therapeutically effective doses of the anti-cancer monoclonal antibody in combination with the administration of one or more therapeutically effective doses of IL-2 or biologically active variant thereof.

The allelic pattern of the individual can be detected using any detection method known in the art, including, but not limited to, testing blood cells or DNA from the individual for the presence of the different FcγRIIIA and/or FcγRIIA allelic variants using antibody-based and/or nucleic acid-based diagnostics described further herein below. In one embodiment, the allelic pattern is detected by determining whether each copy of the FcγRIIIA gene in a DNA sample obtained from the individual contains a T or a G at position 526 of the FcγRIIIA coding region shown in SEQ ID NO:1 and/or whether the FcγRIIIA polypeptides expressed at the surface of immune cells of the individual contain the corresponding valine or phenylalanine residue at position 158 of the mature human FcγRIIIA (i.e., at position 176 of the full-length translated product shown in SEQ ID NO:2). In another embodiment, the allelic pattern is detected by determining whether each copy of the FcγRIIA gene in a DNA sample obtained from the individual contains a G or an A at position 494 of the FcγRIIA coding region shown in SEQ ID NO:3 and/or whether the FcγRIIA polypeptides expressed at the surface of immune cells of the individual contain the corresponding histidine or arginine residue at position 131 of mature human FcγRIIA (i.e., at position 165 of the full-length translated product shown in SEQ ID NO:4).

Methods for detecting the allelic pattern of the FcγRIIIA and FcγRIIA genes are well known in the art. See for example, the genotyping methods disclosed in Koene et al. (1997) Blood 90:1109-1114 (nested PCR-based allele-specific restriction analysis assay for detection of FcγRIIIA genotype) and Jiang et al. (1996) J. Immunol. Methods 199:55-59 (PCR-based allele-specific restriction enzyme digestion for detection of FcγRIIA genotype); Morgan et al. (2003) Rheumatology 42:528-533 (single-stranded conformational polymorphism assay for detection of FcγRIIIA genotype); Dall'Ozzo et al. (2003) J. Immunol. Methods 277:185-192 (real-time multiplex allele-specific PCR and melting curve analysis in the presence of SYBR Green I fluorescent dye for detection of FcγRIIIA genotype); and U.S. Pat. Nos. 5,830,652 and 5,985,561 (detection of FcγRIIA or FcγRIIIA phenotype by flow cytometry, genotyping using PCR-based allele-specific restriction analysis assay, and single-stranded conformational polymorphism); de Haas et al. (1996) J. Immunology 156(8):3948 (detection of FcγRIIIA 48 L/R/H genotype); each of which is herein incorporated by reference in its entirety.

In one embodiment of the invention, the FcγRIIA or FcγRIIIA genotype (i.e., allelic pattern) in an individual is determined by either: 1) immunological detection of one or more allelic forms of FcγRIIA or FcγRIIIA polypeptides present on the surface of appropriate immune cells (i.e., “phenotypic characterization”); or 2) molecular detection of the DNA or RNA encoding one or more FcγRIIA or FcγRIIIA allelic forms using nucleic acid probes, with or without nucleic acid amplification or sequencing (i.e., “genotypic characterization”).

In the first method, white blood cells or subsets thereof are isolated from an individual to be tested using methods that are well known in the art, such as, for example, gradient centrifugation and/or immunoadsorption. Antibodies that are capable of distinguishing between different allelic forms of FcγRIIA or FcγRIIIA are then applied to the isolated cells to determine the presence and relative amount of each allelic form. The antibodies may be polyclonal or monoclonal, preferably monoclonal. Measurement of specific antibody binding to cells may be accomplished by any known method, including without limitation quantitative flow cytometry, or enzyme-linked or fluorescence-linked immunoassay. The presence or absence of a particular allele, as well as the allelic pattern (i.e., homozygosity vs. heterozygosity) is determined by comparing the values obtained from the individual with norms established from populations of individuals of known gentoypes.

In an alternate embodiment, a DNA sample is obtained from an individual, and the presence of DNA sequences corresponding to particular FcγRIIA or FcγRIIIA alleles is determined. The DNA may be obtained from any cell source or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, and tissue exudates at the site of the biopsy. DNA is extracted from the cell source or body fluid using any of the numerous methods that are standard in the art. It will be understood that the particular method used to extract DNA will depend on the nature of the source. In some embodiments, the cell source or body fluid is PMBC or serum.

Once extracted, the DNA may be employed in the present invention without further manipulation. Alternatively, the DNA region corresponding to all or part of the FcγRIIA or FcγRIIIA may be amplified by PCR or other amplification methods known in the art. In this case, the amplified regions are specified by the choice of particular flanking sequences for use as primers. Amplification at this step provides the advantage of increasing the concentration of FcγRIIA or FcγRIIIA DNA sequences. The length of DNA sequence that can be amplified ranges from 80 bp to up to 30 kbp. Preferably, primers are used that define a relatively short segment containing sequences that differ between different allelic forms of the respective receptors. A preferred detection method is allele-specific hybridization using probes overlapping the polymorphic site of interest (i.e., FcγRIIA 131H or R allele; FcγRIIIA 158V or F allele; or FcγRIIIA 48L, R, or H allele) and having about 5, 10, 15, 20, 25, or 30 nucleotides around the polymorphic region.

The presence of FcγRIIA or FcγRIIIA allele-specific DNA sequences may be determined by any known method, including without limitation direct DNA sequencing, hybridization with allele-specific oligonucleotides, and single-stranded conformational polymorphism (SSCP). Direct sequencing may be accomplished by chemical sequencing, for example, using the Maxam-Gilbert method, or by enzymatic sequencing, for example, using the Sanger method. In the latter case, specific oligonucleotides are synthesized using standard methods and used as primers for the dideoxynucleotide sequencing reaction.

Preferably, DNA from an individual is subjected to amplification by polymerase chain reaction (PCR) using specific amplification primers, followed by hybridization with allele-specific oligonucleotides. Alternatively, SSCP analysis of the amplified DNA regions may be used to determine the allelic pattern. Most preferably, allele-specific PCR is used, in which allele-specific oligonucleotides are used as primers and the presence or absence of an amplification product indicates the presence or absence of a particular allele.

In an alternate embodiment, cells expressing FcγRIIA or FcγRIIIA are isolated by immunoadsorption, and RNA is isolated from the immunopurified cells using well-known methods such as guanidium thiocyanate-phenol-chloroform extraction (Chomocyznski et al. (1987) Anal. Biochem. 162:156). The isolated RNA is then subjected to coupled reverse transcription and amplification by polymerase chain reaction (RT-PCR), using allele-specific oligonucleotide primers. Conditions for primer annealing are chosen to ensure specific reverse transcription and amplification; thus, the appearance of an amplification product is diagnostic of the presence of the allele specified by the particular primer employed. In another embodiment, RNA encoding FcγRIIA or FcγRIIIA is reverse-transcribed and amplified in an allele-independent manner, after which the amplified FcγRIIA- or FcγRIIIA-encoding cDNA is identified by hybridization to allele-specific oligonucleotides or by direct DNA sequencing. For allele-specific primers for the FcγRIIA gene, see, for example, the references cited above wherein PCR-based methods are utilized to detect the presence or absence of particular FcγRIIA or FcγRIIIA alleles.

In one embodiment, the genotype of the subject is determined as described in co-owned U.S. Ser. No. 60/560,649, “Nucleic Acid Based Assays For Identification Of Fc Receptor Polymorphisms,” filed Apr. 7, 2004 and incorporated by reference herein in its entirety.

Individuals in need of treatment for an immune disorder and who are identified as carriers of the FcγRIIIA 158 F/F genotype; the FcγRIIIA 48 L/L genotype, FcγRIIIA 48 L/R genotype, or FcγRIIIA 48 L/H genotype; and/or the FcγRIIA 131 H/R or FcγRIIA 131 R/R genotype are suitable candidates for intervention with IL-2 immunotherapy as defined herein above. Thus, the present invention also provides methods for enhancing the immune function of an individual that is a carrier of the FcγRIIIA 158F/F genotype and/or the FcγRIIIA 48 L/L genotype, FcγRIIIA 48 L/R genotype, or FcγRIIIA 48 L/H genotype, and/or the FcγRIIA 131 H/R or FcγRIIA 131 R/R genotype, and for treating such an individual for an immune disorder. The methods comprise administering IL-2 immunotherapy to such an individual. As previously noted, the IL-2 immunotherapy can be the sole line of treatment; alternatively, the individual can be undergoing treatment with another agent, particularly an agent that mediates its therapeutic effect via its interaction with FcγRIIIA or FcγRIIA and the ADCC pathway triggered by this interaction. In one embodiment, the individual is suffering from an immune disorder, particularly a cancer, and is administered IL-2 immunotherapy alone or in combination with an anti-cancer monoclonal antibody. Examples of cancers that can be treated using the methods of the present invention include, but are not limited to, B-cell lymphomas listed below, breast cancer, ovarian cancer, cervical cancer, prostate cancer, colon cancers, melanoma, renal cell carcinoma, acute myeloid leukemia (AML); and chronic lymphocytic leukemia (CLL). As noted above, the individual is administered one or more therapeutically effective doses of the anti-cancer monoclonal antibody in combination with the administration of one or more therapeutically effective doses of IL-2 or biologically active variant thereof.

Combination IL-2 immunotherapy and anti-cancer monoclonal antibody therapy provides for anti-tumor activity. By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Subjects undergoing therapy with a combination of IL-2 immunotherapy and at least one anti-cancer monoclonal antibody in accordance with the methods of the present invention experience a physiological response that is beneficial with respect to treatment of a particular cancer of interest.

The separate pharmaceutical compositions comprising the therapeutic agent or agents used in the cancer therapy protocol and the IL-2 or variant thereof may be administered using the same or different routes of administration in accordance with any medically acceptable method known in the art. Suitable routes of administration include parenteral administration, such as subcutaneous (SC), intramuscular (IM), intravenous (IV), or infusion, oral and pulmonary, nasal, topical, transdermal, and suppositories. Where IL-2 or variant thereof is administered via pulmonary delivery,. the therapeutically effective dose is adjusted such that the soluble level of IL-2 or variant thereof in the bloodstream is equivalent to that obtained with a therapeutically effective dose that is administered parenterally, for example SC, IM, or IV. Preferably the pharmaceutical composition comprising IL-2 or variant thereof is administered by any form of injection, including intravenous (IV), intramuscular (IM), or subcutaneous (SC) injection. In some embodiments of the invention, the pharmaceutical composition comprising IL-2 or variant thereof is administered by IM or SC injection, particularly by IM or SC injection locally to the region where the therapeutic agent or agents used in the cancer therapy protocol are administered. Where IL-2 immunotherapy is being administered concurrently with another agent, particularly an anti-cancer monoclonal antibody or antigen-binding fragment thereof, the pharmaceutical composition comprising this agent is administered, for example, intravenously. When administered intravenously, the pharmaceutical composition comprising the anti-cancer monoclonal antibody or antigen-binding fragment thereof can be administered by infusion over a period of about 0.5 to about 5 hours. In some embodiments, infusion occurs over a period of about 0.5 to about 2.5 hours, over a period of about 0.5 to about 2.0 hours, over a period of about 0.5 to about 1.5 hours, or over a period of about 1.5 hours, depending upon the anti-cancer monoclonal antibody being administered and the amount of anti-cancer monoclonal antibody being administered.

Factors influencing the respective amount of IL-2 or variant thereof to be administered during the course of IL-2 immunotherapy include, but are not limited to, the mode of administration, the frequency of administration (i.e., daily, or intermittent administration, such as twice- or thrice-weekly), the particular disease undergoing therapy, the severity of the disease, the history of the disease, whether the individual is undergoing concurrent therapy with another therapeutic agent, for example, an anti-cancer monoclonal antibody, and the age, height, weight, health, and physical condition of the individual undergoing therapy. Generally, a higher dosage of this agent is preferred with increasing weight of the subject undergoing therapy.

In one embodiment of the invention, the individual carrying the FcγRIIIA 158F/F genotype and/or the FcγRIIA 131H/R or FcγRIIA 131R/R genotype, undergoes combination IL-2 immunotherapy and anti-CD20 antibody therapy for a B-cell lymphoma, more particularly non-Hodgkin's B-cell lymphoma. The therapeutic methods of the invention are directed to treatment of any non- Hodgkin's B-cell lymphoma whose abnormal B-cell type expresses the CD20 surface antigen. By “CD20 surface antigen” is intended a 33-37 kD integral membrane phosphoprotein that is expressed during early pre-B cell development and persists through mature B-cells but which is lost at the plasma cell stage. Although CD20 is expressed on normal B cells, this surface antigen is usually expressed at very high levels on neoplastic B cells. More than 90% of B-cell lymphomas and chronic lymphocytic leukemias, and about 50% of pre-B-cell acute lymphoblastic leukemias express this surface antigen.

It is recognized that concurrent therapy with IL-2 immunotherapy and an anti-CD20 antibody may be useful in the treatment of any type of cancer whose unabated proliferating cells express the CD20 surface antigen. Thus, for example, where a cancer is associated with aberrant T-cell proliferation, and the aberrant T-cell population expresses the CD20 surface antigen, concurrent therapy in accordance with the methods of the invention would provide a positive therapeutic response with respect to treatment of that cancer. A human T-cell population expressing the CD20 surface antigen, though in reduced amounts relative to B-cells, has been identified (see Hultin et al. (1993) Cytometry 14:196-204).

It also is recognized that the methods of the invention are useful in the therapeutic treatment of B-cell lymphomas that are classified according to the Revised European and American Lymphoma Classification (REAL) system. Such B-cell lymphomas include, but are not limited to, lymphomas classified as precursor B-cell neoplasms, such as B-lymphoblastic leukemia/lymphoma; peripheral B-cell neoplasms, including B-cell chronic lymphocytic leukemia/small lymphocytic lymphoma, lymphoplasmacytoid lymphoma/immunocytoma, mantle cell lymphoma (MCL), follicle center lymphoma (follicular) (including diffuse small cell, diffuse mixed small and large cell, and diffuse large cell lymphomas), marginal zone B-cell lymphoma (including extranodal, nodal, and splenic types), hairy cell leukemia, plasmacytoma/ myeloma, diffuse large cell B-cell lymphoma of the subtype primary mediastinal (thymic), Burkitt's lymphoma, and Burkitt's like high grade B-cell lymphoma; and unclassifiable low-grade or high-grade B-cell lymphomas.

By “non-Hodgkin's B-cell lymphoma” is intended any of the non-Hodgkin's based lymphomas related to abnormal, uncontrollable B-cell proliferation. For purposes of the present invention, such lymphomas are referred to according to the Working Formulation classification scheme (see “The Non-Hodgkin's Lymphoma Pathologic Classification Project,” Cancer 49(1982):2112-2135), that is those B-cell lymphomas categorized as low grade, intermediate grade, and high grade. Low-grade B-cell lymphomas include small lymphocytic, follicular small-cleaved cell, and follicular mixed small-cleaved cell lymphomas; intermediate-grade lymphomas include follicular large cell, diffuse small cleaved cell, diffuse mixed small and large cell, and diffuse large cell lymphomas; and high-grade lymphomas include large cell immunoblastic, lymphoblastic, and small non-cleaved cell lymphomas of the Burkitt's and non-Burkitt's type.

While the methods of the invention are directed to treatment of an existing lymphoma or solid tumor, it is recognized that the methods may be useful in preventing further tumor outgrowths arising during therapy. The methods of the invention are particularly useful in the treatment of subjects having low-grade B-cell lymphomas, particularly those subjects having relapses following standard chemotherapy. Low-grade B-cell lymphomas are more indolent than the intermediate- and high-grade B-cell lymphomas and are characterized by a relapsing/remitting course. Thus, treatment of these lymphomas is improved using the methods of the invention, as relapse episodes are reduced in number and severity.

Particular treatment protocols for IL-2 immunotherapy in combination with anti-cancer monoclonal antibodies are known in the art. Such protocols can be utilized to treat an individual that has been identified as a carrier of the FcγRIIIA 158F/F genotype; and/or the FcγRIIIA 48L/L, or FcγRIIIA 48 L/R, or FcγRIIIA 48L/H genotype; and/or the FcγRIIA 131H/R or FcγRIIA 131R/R genotype. See, for example, the treatment protocols disclosed in copending U.S. Patent Publication 2003-0185796 (B-cell lymphomas) and copending U.S. patent application Ser. No. 60/491,371, entitled “Methods of Therapy for Chronic Lymphocytic Leukemia,” Attorney Docket No. 59516-278, filed Jul. 31, 2003; the contents of which are herein incorporated by reference in their entirety. The amount of IL-2 (either native-sequence or variant thereof retaining IL-2 biological activity, such as muteins disclosed herein) administered may range between about 0.1 to about 15 mIU/m². For indications such as renal cell carcinoma and metastatic melanoma, the IL-2 or biologically active variant thereof may be administered as a high-dose intravenous bolus at 300,000 to 800,000 IU/kg/8hours. See the foregoing U.S. patent applications for recommended doses for IL-2 immunotherapy for B-cell lymphomas and CLL.

Where an individual having the FcγRIIIA 158F/F genotype; and/or the FcγRIIIA 48L/L, or FcγRIIIA 48 L/R, or FcγRIIIA 48L/H genotype; and/or the FcγRIIA 131H/R or FcγRIIA 131R/R genotype is undergoing treatment with IL-2 immunotherapy and an anti-cancer monoclonal antibody, these therapeutic agents are presented to the individual by way of concurrent therapy. By “concurrent therapy” is intended presentation of IL-2 and at least one anti-cancer antibody to a human subject such that the therapeutic effect of the combination of both substances is caused in the subject undergoing therapy. Concurrent therapy may be achieved by administering at least one therapeutically effective dose of a pharmaceutical composition comprising IL-2 or variant thereof and at least one therapeutically effective dose of a pharmaceutical composition comprising at least one anti-cancer antibody or antigen-binding fragment thereof according to a particular dosing regimen. Administration of the separate pharmaceutical compositions can be at the same time (i.e., simultaneously) or at different times (i.e., sequentially, in either order, on the same day, or on different days), so long as the therapeutic effect of the combination of both substances is caused in the subject undergoing therapy.

The separate pharmaceutical compositions comprising these therapeutic agents as therapeutically active components may be administered using any acceptable method known in the art. Thus, for example, the pharmaceutical composition comprising IL-2 or variant thereof can be administered by any form of injection, including intravenous (IV), intramuscular (IM), or subcutaneous (SC) injection. In some embodiments of the invention, the pharmaceutical composition comprising IL-2 or variant thereof is administered by SC injection. In other embodiments of the invention, the pharmaceutical composition comprising IL-2 or variant thereof is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition comprising IL-2 or variant thereof. The pharmaceutical composition comprising the anti-cancer antibody or antigen-binding fragment thereof is administered, for example, intravenously. When administered intravenously, the pharmaceutical composition comprising the anti-cancer antibody can be administered by infusion over a period of about 1 to about 10 hours. In some embodiments, infusion of the antibody occurs over a period of about 2 to about 8 hours, over a period of about 3 to about 7 hours, over a period of about 4 to about 6 hours, or over a period of about 6 hours, depending upon the anti-cancer antibody being administered.

Where a subject undergoing therapy in accordance with the previously mentioned dosing regimens exhibits a partial response, or a relapse following a prolonged period of remission, subsequent courses of concurrent therapy may be needed to achieve complete remission of the disease. Thus, subsequent to a period of time off from a first treatment period, a subject may receive one or more additional treatment periods comprising IL immunotherapy combination with anti-cancer antibody administration. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these two therapeutic agents.

The term “IL-2” as used herein refers to a lymphokine that is produced by normal peripheral blood lymphocytes and is present in the body at low concentrations. IL-2 was first described by Morgan et al. (1976) Science 193:1007-1008 and originally called T cell growth factor because of its ability to induce proliferation of stimulated T lymphocytes. It is a protein with a reported molecular weight in the range of 13,000 to 17,000 (Gillis and Watson (1980) J. Exp. Med. 159:1709) and has an isoelectric point in the range of 6-8.5.

The IL-2 present in the pharmaceutical compositions described herein for use in the methods of the invention may be native or obtained by recombinant techniques, and may be from any source, including mammalian sources such as, e.g., mouse, rat, rabbit, primate, pig, and human. IL-2 sequences from a number of species are well known in the art. See, for example, but not limited to, the following: human IL-2 (Homo sapiens; precursor sequence, GenBank Accession No. AAH66254; mature sequence represented by residues 21-153 of GenBank Accession No. AAH66254 sequence and set forth in SEQ ID NO:14 herein); rhesus monkey IL-2 (Macaca mulatto; precursor sequence, GenBank Accession No. P51498; mature sequence represented by residues 21-154 of GenBank Accession No. P51498 sequence); olive baboon IL-2 (Papio anubis; precursor sequence, GenBank Accession No. Q865Y1; mature sequence represented by residues 21-154 of GenBank Accession No. Q865Y1 sequence); sooty mangabey IL-2 (Cercocebus torquatus atys; precursor sequence, GenBank Accession No. P46649; mature sequence represented by residues 21-154 of GenBank Accession No. P46649 sequence); crab-eating macaque IL-2 (Macaca fascicularis; precursor sequence, GenBank Accession No. Q29615; mature sequence represented by residues 21-154 of GenBank Accession No. Q29615 sequence); common gibbon IL-2 (Hylobates lar; precursor sequence, GenBank Accession No. ICG12; mature sequence represented by residues 21-153 of GenBank Accession No. ICG12 sequence); common squirrel monkey IL-2 (Saimiri sciureus; precursor sequence, GenBank Accession No. Q8MKH2; mature sequence represented by residues 21-154 of GenBank Accession No. Q8MKH2 sequence); cow IL-2 (Bos taurus; precursor sequence, GenBank Accession No. P05016; mature sequence represented by residues 21-155 of GenBank Accession No. P05016 sequence; see also the variant precursor sequence reported in GenBank Accession No. NP-851340; mature sequence represented by residues 24-158 of GenBank Accession No. NP-851340 sequence); water buffalo IL-2 (Bubalus bubalis; precursor sequence, GenBank Q95KP3; mature sequence represented by residues 21-155 of GenBank Q9SKP3 sequence); horse IL-2 (Equus caballus; precursor sequence, GenBank Accession No. P37997; mature sequence represented by residues 21-149 of GenBank Accession No. P37997 sequence); goat IL-2 (Capra hircus; precursor sequence, GenBank Accession No. P36835; mature sequence represented by residues 21-155 of GenBank Accession No. P36835 sequence); sheep IL-2 (Ovis aries; precursor sequence, GenBank Accession No. P19114; mature sequence represented by residues 21-155 of GenBank Accession No. P19114 sequence); pig IL-2 (Sus scrofa; precursor sequence, GenBank Accession No. P26891; mature sequence represented by residues 21-154 of GenBank Accession No. P26891); red deer IL-2 (Cervus elaphus; precursor sequence, GenBank Accession No. P51747; mature sequence represented by residues 21-162 of GenBank Accession No. P51747 sequence); dog IL-2 (Canis familiaris; precursor sequence, GenBank Accession No. Q29416; mature sequence represented by residues 21-155 of GenBank Accession No. Q29416 sequence); cat IL-2 (Felis catus; precursor sequence, GenBank Accession No. Q07885; mature sequence represented by residues 21-154 of GenBank Accession No. Q07885 sequence); rabbit IL-2 (Oryctolagus cuniculus; precursor sequence, GenBank Accession No. O77620; mature sequence represented by residues 21-153 of GenBank Accession No. O77620 sequence); killer whale IL-2 (Orcinus orca; precursor sequence, GenBank Accession No. O97513; mature sequence represented by residues 21-152 of GenBank Accession No. O97513 sequence); northern elephant seal IL-2 (Mirounga angustirostris; precursor sequence, GenBank Accession No. O62641; mature sequence represented by residues 21-154 of GenBank Accession No. O62641 sequence); house mouse IL-2 (Mus musculus; precursor sequence, GenBank Accession No. NP_{—}032392; mature sequence represented by residues 21-169 of GenBank Accession No. NP_{—}032392 sequence); western wild mouse IL-2 (Mus spretus; precursor sequence, GenBank Accession No. Q08867; mature sequence represented by residues 21-166 of GenBank Accession No. Q08867 sequence); Norway rat IL-2 (Rattus norvegicus; precursor sequence, GenBank Accession No. P17108; mature sequence represented by residues 21-155 of GenBank Accession No. P17108); Mongolian gerbil IL-2 (Meriones unguiculatus; precursor sequence, GenBank Accession No. Q08081; mature sequence represented by residues 21-155 of GenBank Accession No. Q08081); any of the variant IL-2 polypeptides disclosed in these foregoing GenBank Accession Numbers; each of which GenBank reports are herein incorporated by reference in their entirety. Though any source of IL-2 can be utilized to practice the invention, preferably the IL-2 is derived from a human source, particularly when the subject undergoing therapy is a human. In some embodiments, the IL-2 for use in the methods of the invention is recombinantly produced, for example, recombinant human IL-2 proteins, including, but not limited to, those obtained from microbial hosts.

The pharmaceutical compositions useful in the methods of the invention may comprise biologically active variants of IL-2, including variants of IL-2 from any species. Such variants should retain the desired biological activity of the native polypeptide such that the pharmaceutical composition comprising the variant polypeptide has the same therapeutic effect as the pharmaceutical composition comprising the native polypeptide when administered to a subject. That is, the variant polypeptide will serve as a therapeutically active component in the pharmaceutical composition in a manner similar to that observed for the native polypeptide. Methods are available in the art for determining whether a variant polypeptide retains the desired biological activity, and hence serves as a therapeutically active component in the pharmaceutical composition. Biological activity can be measured using assays specifically designed for measuring activity of the native polypeptide or protein, including assays described in the present invention. Additionally, antibodies raised against a biologically active native polypeptide can be tested for their ability to bind to the variant polypeptide, where effective binding is indicative of a polypeptide having a conformation similar to that of the native polypeptide.

Suitable biologically active variants of native or naturally occurring IL-2 can be fragments, analogues, and derivatives of that polypeptide. By “fragment” is intended a polypeptide consisting of only a part of the intact polypeptide sequence and structure, and can be a C-terminal deletion or N-terminal deletion of the native polypeptide. By “analogue” is intended an analogue of either the native polypeptide or of a fragment of the native polypeptide, where the analogue comprises a native polypeptide sequence and structure having one or more amino acid substitutions, insertions, or deletions. “Muteins”, such as those described herein, and peptides having one or more peptoids (peptide mimics) are also encompassed by the term analogue (see International Publication No. WO 91/04282). See, also, U.S. Ser. No. 60/585,980, filed Jul. 7, 2004 and titled “Combinatorial Interleukin-2 Muteins;” as well as U.S. Ser. No. 60/550,868, filed Mar. 5, 2004, and titled “Improved Interleukin-2 Muteins;” which applications are incorporated by reference herein in their entireties.

By “derivative” is intended any suitable modification of the native polypeptide of interest, of a fragment of the native polypeptide, or of their respective analogues, such as glycosylation, phosphorylation, polymer conjugation (such as with polyethylene glycol), or other addition of foreign moieties, so long as the desired biological activity of the native polypeptide is retained. Methods for making polypeptide fragments, analogues, and derivatives are generally available in the art.

For example, amino acid sequence variants of the polypeptide can be prepared by mutations in the cloned DNA sequence encoding the native polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor Laboratory Press, Plainview, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly⇄Ala, Val⇄Ile⇄Leu, Asp⇄Glu, Lys⇄Arg, Asn⇄Gln, and Phe⇄Trp⇄Tyr.

Guidance as to regions of the IL-2 protein that can be altered either via residue substitutions, deletions, or insertions can be found in the art. See, for example, the structure/function relationships and/or binding studies discussed in Bazan (1992) Science 257:410-412; McKay (1992) Science 257:412; Theze et al. (1996) Immunol. Today 17:481-486; Buchli and Ciardelli (1993) Arch. Biochem. Biophys. 307:411-415; Collins et al. (1988) Proc. Natl. Acad. Sci. USA 85:7709-7713; Kuziel et al. (1993) J. Immunol. 150:573 1; Eckenberg et al. (1997) Cytokine 9:488-498; the contents of which are herein incorporated by reference in their entirety.

In constructing variants of the IL-2 polypeptide of interest, modifications are made such that variants continue to possess the desired activity. Obviously, any mutations made in the DNA encoding the variant polypeptide must not place the sequence out of reading frame and preferably will not create complementary regions that could produce secondary MRNA structure. See EP Patent Application Publication No. 75,444.

Biologically active variants of IL-2 will generally have at least about 70%, preferably at least about 80%, more preferably at least about 90% to 95% or more, and most preferably at least about 98%, 99% or more amino acid sequence identity to the amino acid sequence of the reference IL-2 polypeptide molecule, such as native human IL-2, which serves as the basis for comparison. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman, Adv. Appl. Math. (1981) 2:482-489. A variant may, for example, differ by as few as 1 to 15 amino acid residues, as few as 1 to 10 residues, such as 6-10, as few as 5, as few as 4, 3, 2, or even 1 amino acid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm). A biologically active variant of a native IL-2 polypeptide of interest may differ from the native polypeptide by as few as 1-15 amino acids, as few as 1-10, such as 6-10, as few as 5, as few as 4, 3, 2, or even 1 amino acid residue.

The precise chemical structure of a polypeptide having IL-2 activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of polypeptides having IL-2 activity as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an IL-2 polypeptide used herein so long as the IL-2 activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of IL-2 polypeptides of interest as used herein.

The art provides substantial guidance regarding the preparation and use of polypeptide variants. In preparing the IL-2 variants, one of skill in the art can readily determine which modifications to the native protein nucleotide or amino acid sequence will result in a variant that is suitable for use as a therapeutically active component of a pharmaceutical composition used in the methods of the present invention.

The IL-2 or variants thereof for use in the methods of the present invention may be from any source, but preferably is recombinantly produced. By “recombinant IL-2” or “recombinant IL-2 variant” is intended interleukin-2 or variant thereof that has comparable biological activity to native-sequence IL-2 and that has been prepared by recombinant DNA techniques as described, for example, by Taniguchi et al. (1983) Nature 302:305-310 and Devos (1983) Nucleic Acids Research 11:4307-4323 or mutationally altered IL-2 as described by Wang et al. (1984) Science 224:1431-1433. In general, the gene coding for IL-2 is cloned and then expressed in transformed organisms, preferably a microorganism, and most preferably E. coli, as described herein. The host organism expresses the foreign gene to produce IL-2 under expression conditions. Synthetic recombinant IL-2 can also be made in eukaryotes, such as yeast or human cells. Processes for growing, harvesting, disrupting, or extracting the IL-2 from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

For examples of variant IL-2 proteins, see European Patent (EP) Publication No. EP 136,489 (which discloses one or more of the following alterations in the amino acid sequence of naturally occurring IL-2: Asn26 to Gln26; Trp12l to Phe12l; Cys58 to Ser58 or Ala58, Cys105 to Ser105 or Ala105; Cys125 to Ser125 or Ala125; deletion of all residues following Arg 120; and the Met-1 forms thereof); and the recombinant IL-2 muteins described in European Patent Application No. 83306221.9, filed Oct. 13, 1983 (published May 30, 1984 under Publication No. EP 109,748), which is the equivalent to Belgian Patent No. 893,016, and commonly owned U.S. Pat. No. 4,518,584 (which disclose recombinant human IL-2 mutein wherein the cysteine at position 125, numbered in accordance with native human IL-2, is deleted or replaced by a neutral amino acid; alanyl-ser125-IL-2; and des-alanayl-ser125-IL-2). See also U.S. Pat. No. 4,752,585 (which discloses the following variant IL-2 proteins: ala104 ser125 IL-2, ala104 IL-2, ala104 ala125 IL-2, val104 ser125 IL-2, val104 IL-2, val104 ala125 IL-2, des-ala1 ala104 ser125 IL-2, des-ala1 ala104 IL-2, des-ala1 ala104 ala125 IL-2, des-ala1 val104 ser125 IL-2, des-ala1 val104 IL-2, des-ala1 val104 ala125 IL-2, des-ala1 des-pro2 ala104 ser125 IL-2, des-ala1 des-pro2 ala104 IL-2, des-ala1 des-pro2 ala104 ala125 IL-2, des-ala1 des-pro2 val104 ser125 IL-2, des-ala1 des-pro2 val104 IL-2, des-ala1 des-pro2 val104 ala125 IL-2, des-ala1 des-pro2 des-thr3 ala104 ser125 IL-2, des-ala1 des-pro2 des-thr3 ala104 IL-2, des-ala1 des-pro2 des-thr3 ala104 ala125 IL-2, des-ala1 des-pro2 des-thr3 val104 ser125 IL-2, des-ala1 des-pro2 des-thr3 val104 IL-2, des-ala1 des-pro2 des-thr3 val104 ala125 IL-2, des-ala1 des-pro2 des-thr3 des-ser4 ala104 ser125 IL-2, des-ala1 des-pro2 des-thr3 des-ser4 ala104 IL-2, des-ala1 des-pro2 des-thr3 des-ser4 ala104 ala125 IL-2, des-ala1 des-pro2 des-thr3 des-ser4 val104 ser125 IL-2, des-ala1 des-pro2 des-thr3 des-ser4 val104 IL-2, des-ala1 des-pro2 des-thr3 des-ser4 val104 ala125 IL-2, des-ala1 des-pro2 des-thr3 des-ser4 des-ser5 ala104 ser125 IL-2, des-ala1 des-pro2 des-thr3 des-ser4 des-ser5 ala104 IL-2, des-ala1 des-pro2 des-thr3 des-ser4 des-ser5 ala104 ala125 IL-2, des-ala1 des-pro2 des-thr3 des-ser4 des-ser5 val104 ser125 IL-2, des-ala1 des-pro2 des-thr3 des-ser4 des-ser5 val104 IL-2, des-ala1 des-pro2 des-thr3 des-ser4 des-ser5 val104 ala125 IL-2, des-ala1 des-pro2 des-thr3 des-ser4 des-ser5 des-ser6 ala104 ala125 IL-2, des-ala1 des-pro2 des-thr3 des-ser4 des-ser5 des-ser6 ala104 IL-2, des-ala1 des-pro2 des-thr3 des-ser4 des-ser5 des-ser6 ala104 ser125 IL-2, des-ala1 des-pro2 des-thr3 des-ser4 des-ser5 des-ser6 val104 ser125 IL-2, des-ala1 des-pro2 des-thr3 des-ser4 des-ser5 des-ser6 val104 IL-2, and des-ala1 des-pro2 des-thr3 des-ser4 des-ser5 des-ser6 val104 ala125 IL-2 ) and U.S. Pat. No. 4,931,543 (which discloses the IL-2 mutein des-alanyl-1, serine-125 human IL-2 used in the examples herein, as well as the other IL-2 muteins).

Also see European Patent Publication No. EP 200,280 (published Dec. 10, 1986), which discloses recombinant IL-2 muteins wherein the methionine at position 104 has been replaced by a conservative amino acid. Examples include the following muteins: ser4 des-ser5 ala104 IL-2; des-ala1 des-pro2 des-thr3 des-ser4 des-ser5 ala104 ala125 IL-2; des-ala1 des-pro2 des-thr3 des-ser4 des-ser5 glu104 ser125 IL-2; des-ala1 des-pro2 des-thr3 des-ser4 des-ser5 glu104 IL-2; des-ala1 des-pro2 des-thr3 des-ser4 des-ser5 glu104 ala125 IL-2; des-ala1 des-pro2 des-thr3 des-ser4 des-ser5 des-ser6 ala104 ala125 IL-2; des-ala1 des-pro2 des-thr3 des-ser4 des-ser5 des-ser6 ala104 IL-2; des-ala1 des-pro2 des-thr3 des-ser4 des-ser5 des-ser6 ala104 ser125 IL-2; des-ala1 des-pro2 des-thr3 des-ser4 des-ser5 des-ser6 glu104 ser125 IL-2; des-ala1 des-pro2 des-thr3 des-ser4 des-ser5 des-ser6 glu104 IL-2; and des-ala1 des-pro2 des-thr3 des-ser4 des-ser5 des-ser6 glu104 ala125 IL-2. See also European Patent Publication No. EP 118,617 and U.S. Pat. No. 5,700,913, which disclose unglycosylated human IL-2 variants bearing alanine instead of native IL-2's methionine as the N-terminal amino acid; an unglycosylated human IL-2 with the initial methionine deleted such that proline is the N-terminal amino acid; and an unglycosylated human IL-2 with an alanine inserted between the N-terminal methionine and proline amino acids.

Other IL-2 muteins include the those disclosed in WO 99/60128 (substitutions of the aspartate at position 20 with histidine or isoleucine, the asparagine at position 88 with arginine, glycine, or isoleucine, or the glutamine at position126 with leucine or glutamic acid), which reportedly have selective activity for high affinity IL-2 receptors expressed by cells expressing T cell receptors in preference to NK cells and reduced IL-2 toxicity; the muteins disclosed in U.S Pat. No. 5,229,109 (substitutions of arginine at position 38 with alanine, or substitutions of phenylalanine at position 42 with lysine), which exhibit reduced binding to the high affinity IL-2 receptor when compared to native IL-2 while maintaining the ability to stimulate LAK cells; the muteins disclosed in International Publication No. WO 00/58456 (altering or deleting a naturally occurring (x)D(y) sequence in native IL-2 where D is aspartic acid, (x) is leucine, isoleucine, glycine, or valine, and (y) is valine, leucine or serine), which are claimed to reduce vascular leak syndrome; the IL-2 p1-30 peptide disclosed in International Publication No. WO 00/04048 (corresponding to the first 30 amino acids of IL-2, which contains the entire a-helix A of IL-2 and interacts with the b chain of the IL-2 receptor), which reportedly stimulates NK cells and induction of LAK cells; and a mutant form of the IL-2 p1-30 peptide also disclosed in WO 00/04048 (substitution of aspartic acid at position 20 with lysine), which reportedly is unable to induce vascular bleeds but remains capable of generating LAK cells. Additionally, IL-2 can be modified with polyethylene glycol to provide enhanced solubility and an altered pharmokinetic profile (see U.S. Pat. No. 4,766,106).

Additional examples of IL-2 muteins with predicted reduced toxicity are disclosed in the copending application entitled “Improved IL-2 Muteins,” filed Mar. 5, 2004, and assigned U.S. Provisional Application Serial No. 60/550,868, herein incorporated by reference in its entirety. These muteins comprise the amino acid sequence of mature human IL-2 (SEQ ID NO:14) with a serine substituted for cysteine at position 125 of the mature human IL-2 sequence and at least one additional amino acid substitution within the mature human IL-2 sequence such that the mutein has the following functional characteristics: 1) maintains or enhances proliferation of natural killer (NK) cells, and 2) induces a decreased level of pro-inflammatory cytokine production by NK cells; as compared with a similar amount of des-alanyl-1, C125S human IL-2 or C125S human IL-2 under comparable assay conditions. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, 124L, K32A, K32W, N33E, P34E, P34R, P34S, P34T, P34V, K35D, K35I, K35L, K35M, K35N, K35P, K35Q, K35T, L36A, L36D, L36E, L36F, L36G, L36H, L36I, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M46I, E61K, E61M, E61R, E62T, E62Y, K64D, K64E, K64G, K64L, K64Q, K64R, P65D, P65E, P65F, P65G, P65H, P65I, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L94I, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q1261, and Q126V; where the amino acid residue position is relative to numbering of the mature human IL-2 amino acid sequence (SEQ ID NO:14). In other embodiments, these muteins comprise the amino acid sequence of mature human IL-2 (SEQ ID NO:14) with an alanine substituted for cysteine at position 125 of the mature human IL-2 sequence and at least one additional amino acid substitution within the mature human IL-2 sequence such that the mutein has these same functional characteristics. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, 124L, K32A, K32W, N33E, P34E, P34R, P34S, P34T, P34V, K35D, K35I, K35L, K35M, K35N, K35P, K35Q, K35T, L36A, L36D, L36E, L36F, L36G, L36H, L36I, L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M46I E61K, E61M, E61R, E62T, E62Y, K64D, K64E, K64G, K64L, K64Q, K64R, P65D, P65E, P65F, P65G, P65H, P651, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L941, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q1261, and Q126V; where the amino acid residue position is relative to numbering of the mature human IL-2 amino acid sequence (SEQ ID NO:14). In alternative embodiments, these muteins comprise the amino acid sequence of mature human IL-2 (SEQ ID NO:14) with at least one additional amino acid substitution within the mature human IL-2 sequence such that the mutein has these same functional characteristics. In some embodiments, the additional substitution is selected from the group consisting of T7A, T7D, T7R, K8L, K9A, K9D, K9R, K9S, K9V, K9W, T10K, T10N, Q11A, Q11R, Q11T, E15A, H16D, H16E, L19D, L19E, D20E, 124L, K32A, K32W, N33E, P34E, P34R, P34S, P34T, P34V, K35D, K35I, K35L, K35M, K35N, K35P, K35Q, K35T, L36A, L36D, L36E, L36F, L36G, L36H, L36I L36K, L36M, L36N, L36P, L36R, L36S, L36W, L36Y, R38D, R38G, R38N, R38P, R38S, L40D, L40G, L40N, L40S, T41E, T41G, F42A, F42E, F42R, F42T, F42V, K43H, F44K, M46I, E61K, E61M, E61R, E62T, E62Y, K64D, K64E, K64G, K64L, K64Q, K64R, P65D, P65E, P65F, P65G, P65H, P651, P65K, P65L, P65N, P65Q, P65R, P65S, P65T, P65V, P65W, P65Y, L66A, L66F, E67A, L72G, L72N, L72T, F78S, F78W, H79F, H79M, H79N, H79P, H79Q, H79S, H79V, L80E, L80F, L80G, L80K, L80N, L80R, L80T, L80V, L80W, L80Y, R81E, R81K, R81L, R81M, R81N, R81P, R81T, D84R, S87T, N88D, N88H, N88T, V91A, V91D, V91E, V91F, V91G, V91N, V91Q, V91W, L94A, L94I, L94T, L94V, L94Y, E95D, E95G, E95M, T102S, T102V, M104G, E106K, Y107H, Y107K, Y107L, Y107Q, Y107R, Y107T, E116G, N119Q, T123S, T123C, Q1261, and Q126V; where the amino acid residue position is relative to numbering of the mature human IL-2 amino acid sequence (SEQ ID NO:14). Additional muteins disclosed in this copending application include the foregoing identified muteins, with the exception of having the initial alanine residue at position 1 of the mature human IL-2 sequence deleted.

Additional examples of IL-2 muteins with predicted reduced toxicity are disclosed in the copending application entitled “Combinatorial Interleukin-2 Muteins,” filed Jul. 7, 2004, and assigned U.S. Provisional Application Ser. No. 60/585,980, herein incorporated by reference in its entirety. The combinatorial muteins described in this application include, but are not limited to, a mature human IL-2 amino acid sequence having a serine substituted for cysteine at position 125 and at least two additional amino acid substitutions within the mature human IL-2 sequence such that the mutein has the following functional characteristics: 1) maintains or enhances proliferation of natural killer (NK) cells, and 2) induces a decreased level of pro-inflammatory cytokine production by NK cells; as compared with a similar amount of des-alanyl-1, C125S human IL-2 or C125S human IL-2 under comparable assay conditions, wherein proliferation of NK cells and pro-inflammatory cytokine production are assayed using the NK-92 bioassay. In some embodiments, the mutein further includes a deletion of alanine at position 1. In some embodiments, the additional substitutions are selected from the group consisting of 19D40D, 19D81K, 36D42R, 36D61R, 36D65L, 40D36D, 40D61R, 40D65Y, 40D72N, 40D80K, 40G36D, 40G65Y, 80K36D, 80K65Y, 81K36D, 81K42E, 81K61R, 81K65Y, 81K72N, 81K88D, 81K91D, 81K107H, 81L107H, 91N95G, 107H36D, 107H42E, 107H65Y, 107R36D, 107R72N, 40D81K107H, 40G81K107H, and 91 N94Y95G.

The term IL-2 as used herein is also intended to include IL-2 fusions or conjugates comprising IL-2 fused to a second protein or covalently conjugated to polyproline or a water-soluble polymer to reduce dosing frequencies or to improve IL-2 tolerability. For example, the IL-2 (or a variant thereof as defined herein) can be fused to human albumin or an albumin fragment using methods known in the art (see WO 01/79258). Alternatively, the IL-2 can be covalently conjugated to polyproline or polyethylene glycol homopolymers and polyoxyethylated polyols, wherein the homopolymer is unsubstituted or substituted at one end with an alkyl group and the poplyol is unsubstituted, using methods known in the art (see, for example, U.S. Pat. Nos. 4,766,106, 5,206,344, and 4,894,226).

Any pharmaceutical composition comprising IL-2 as the therapeutically active component can be used in the methods of the invention. Such pharmaceutical compositions are known in the art and include, but are not limited to, those disclosed in U.S. Pat. Nos. 4,745,180; 4,766,106; 4,816,440; 4,894,226; 4,931,544; and 5,078,997; herein incorporated by reference. Thus liquid, lyophilized, or spray-dried compositions comprising IL-2 or variants thereof that are known in the art may be prepared as an aqueous or nonaqueous solution or suspension for subsequent administration to a subject in accordance with the methods of the invention. Each of these compositions will comprise IL-2 or variants thereof as a therapeutically or prophylactically active component. By “therapeutically or prophylactically active component” is intended the IL-2 or variants thereof is specifically incorporated into the composition to bring about a desired therapeutic or prophylactic response with regard to treatment or prevention of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

In preferred embodiments of the invention, the IL-2 containing pharmaceutical compositions useful in the methods of the invention are compositions comprising stabilized monomeric IL-2 or variants thereof, compositions comprising multimeric IL-2 or variants thereof, and compositions comprising stabilized lyophilized or spray-dried IL-2 or variants thereof.

Pharmaceutical compositions comprising stabilized monomeric IL-2 or variants thereof are disclosed in the copending PCT application entitled “Stabilized Liquid 3Polypeptide-Containing Pharmaceutical Compositions,” assigned PCT No. PCTIUSOO/27156, filed Oct. 3, 2000, the disclosure of which is herein incorporated by reference. By “monomeric” IL-2 is intended the protein molecules are present substantially in their monomer form, not in an aggregated form, in the pharmaceutical compositions described herein. Hence covalent or hydrophobic oligomers or aggregates of IL-2 are not present. Briefly, the IL-2 or variants thereof in these liquid compositions is formulated with an amount of an amino acid base sufficient to decrease aggregate formation of IL-2 or variants thereof during storage. The amino acid base is an amino acid or a combination of amino acids, where any given amino acid is present either in its free base form or in its salt form. Preferred amino acids are selected from the group consisting of arginine, lysine, aspartic acid, and glutamic acid. These compositions further comprise a buffering agent to maintain pH of the liquid compositions within an acceptable range for stability of IL-2 or variants thereof, where the buffering agent is an acid substantially free of its salt form, an acid in its salt form, or a mixture of an acid and its salt form. Preferably the acid is selected from the group consisting of succinic acid, citric acid, phosphoric acid, and glutamic acid. Such compositions are referred to herein as stabilized monomeric IL-2 pharmaceutical compositions.

The amino acid base in these compositions serves to stabilize the IL-2 or variants thereof against aggregate formation during storage of the liquid pharmaceutical composition, while use of an acid substantially free of its salt form, an acid in its salt form, or a mixture of an acid and its salt form as the buffering agent results in a liquid composition having an osmolarity that is nearly isotonic. The liquid pharmaceutical composition may additionally incorporate other stabilizing agents, more particularly methionine, a nonionic surfactant such as polysorbate 80, and EDTA, to further increase stability of the polypeptide. Such liquid pharmaceutical compositions are said to be stabilized, as addition of amino acid base in combination with an acid substantially free of its salt form, an acid in its salt form, or a mixture of an acid and its salt form, results in the compositions having increased storage stability relative to liquid pharmaceutical compositions formulated in the absence of the combination of these two components.

These liquid pharmaceutical compositions comprising stabilized monomeric IL-2 or variants thereof may either be used in an aqueous liquid form, or stored for later use in a frozen state, or in a dried form for later reconstitution into a liquid form or other form suitable for administration to a subject in accordance with the methods of present invention. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

Other examples of IL-2 formulations that comprise IL-2 in its nonaggregated monomeric state include those described in Whittington and Faulds (1993) Drugs 46(3):446-514. These formulations include the recombinant IL-2 product in which the recombinant IL-2 mutein Teceleukin (unglycosylated human IL-2 with a methionine residue added at the amino-terminal) is formulated with 0.25% human serum albumin in a lyophilized powder that is reconstituted in isotonic saline, and the recombinant IL-2 mutein Bioleukin (human IL-2 with a methionine residue added at the amino-terminal, and a substitution of the cysteine residue at position 125 of the human IL-2 sequence with alanine) formulated such that 0.1 to 1.0 mg/ml IL-2 mutein is combined with acid, wherein the formulation has a pH of 3.0 to 4.0, advantageously no buffer, and a conductivity of less than 1000 mmhos/cm (advantageously less than 500 mmhos/cm). See EP 373,679; Xhang et al. (1996) Pharmaceut. Res. 13(4):643-644; and Prestrelski et al. (1995) Pharmaceut. Res. 12(9):1250-1258.

Examples of pharmaceutical compositions comprising multimeric IL-2 or variants thereof are disclosed in commonly owned U.S. Pat. No. 4,604,377, the disclosure of which is herein incorporated by reference. By “multimeric” is intended the protein molecules are present in the pharmaceutical composition in a microaggregated form having an average molecular association of 10-50 molecules. These multimers are present as loosely bound, physically-associated IL-2 molecules. A lyophilized form of these compositions is available commercially under the trade name Proleukin (Chiron Corporation, Emeryville, Calif.). The lyophilized formulations disclosed in this reference comprise selectively oxidized, microbially produced recombinant IL-2 in which the recombinant IL-2 is admixed with a water soluble carrier such as mannitol that provides bulk, and a sufficient amount of sodium dodecyl sulfate to ensure the solubility of the recombinant IL-2 in water. These compositions are suitable for reconstitution in aqueous injections for parenteral administration and are stable and well tolerated in human patients. When reconstituted, the IL-2 or variants thereof retains its multimeric state. Such lyophilized or liquid compositions comprising multimeric IL-2 or variants thereof are encompassed by the methods of the present invention. Such compositions are referred to herein as multimeric IL-2 pharmaceutical compositions.

The methods of the present invention may also use stabilized lyophilized or spray-dried pharmaceutical compositions comprising IL-2 or variants thereof, which may be reconstituted into a liquid or other suitable form for administration in accordance with methods of the invention. Such pharmaceutical compositions are disclosed in the copending application entitled “Methods for Pulmonary Delivery of Interleukin-2,” U.S. Ser. No. 09/724,810, filed Nov. 28, 2000 and International Application PCT/US00/35452, filed Dec. 27, 2000, herein incorporated by reference in their entireties. These compositions may further comprise at least one bulking agent, at least one agent in an amount sufficient to stabilize the protein during the drying process, or both. By “stabilized” is intended the IL-2 protein or variants thereof retains its monomeric or multimeric form as well as its other key properties of quality, purity, and potency following lyophilization or spray-drying to obtain the solid or dry powder form of the composition. In these compositions, preferred carrier materials for use as a bulking agent include glycine, mannitol, alanine, valine, or any combination thereof, most preferably glycine. The bulking agent is present in the formulation in the range of 0% to about 10% (w/v), depending upon the agent used. Preferred carrier materials for use as a stabilizing agent include any sugar or sugar alcohol or any amino acid. Preferred sugars include sucrose, trehalose, raffinose, stachyose, sorbitol, glucose, lactose, dextrose or any combination thereof, preferably sucrose. When the stabilizing agent is a sugar, it is present in the range of about 0% to about 9.0% (w/v), preferably about 0.5% to about 5.0%, more preferably about 1.0% to about 3.0%, most preferably about 1.0%. When the stabilizing agent is an amino acid, it is present in the range of about 0% to about 1.0% (w/v), preferably about 0.3% to about 0.7%, most preferably about 0.5%. These stabilized lyophilized or spray-dried compositions may optionally comprise methionine, ethylenediaminetetracetic acid (EDTA) or one of its salts such as disodium EDTA or other chelating agent, which protect the IL-2 or variants thereof against methionine oxidation. Use of these agents in this manner is described in copending U.S. Provisional Application Ser. No. 60/157696, herein incorporated by reference. The stabilized lyophilized or spray-dried compositions may be formulated using a buffering agent, which maintains the pH of the pharmaceutical composition within an acceptable range, preferably between about pH 4.0 to about pH 8.5, when in a liquid phase, such as during the formulation process or following reconstitution of the dried form of the composition. Buffers are chosen such that they are compatible with the drying process and do not affect the quality, purity, potency, and stability of the protein during processing and upon storage.

The previously described stabilized monomeric, multimeric, and stabilized lyophilized or spray-dried IL-2 pharmaceutical compositions represent suitable compositions for use in the methods of the invention. However, any pharmaceutical composition comprising IL-2 or variant thereof as a therapeutically active component is encompassed by the methods of the invention.

As used herein, the term “anti-cancer antibody” encompasses antibodies that have been designed to target cancer cells, particularly cell-surface antigens residing on cells of a particular cancer of interest. Preferably the anti-cancer antibody is monoclonal in nature, and preferably is an IgG1 monoclonal antibody. Suitable IgG1 monoclonal antibodies include, but are not limited to, Rituxan® (which targets the CD20 antigen on neoplastic B cells, and is effective for treatment of B-cell lymphomas, including non-Hodgkin's B-cell lymphomas, and chronic lymphocytic leukemia (CLL)); Therex (humanized HMFG1 specific for MUC1, which is being developed for breast cancer) and other MUC1-positive tumors including ovarian and colon cancers); MDX-010 (human anti-CTLA-4 negative regulator on activated T cells; being developed for melanoma, follicular lymphoma, colon, and prostate cancers); EMD 72000 and Erbitux (IMC-225) (human anti-EGFR being developed for EGFR-positive cancers, most notably colon carcinoma); WX-G250 (specific for MN antigen; being developed for renal cell carcinoma and cervical cancer); IDM-1 (for treatment of ovarian cancer); MDX-210 (for treatment of breast and ovarian cancer); ZAMYL (for treatment of acute myeloid leukemia (AML)); and Campath (for treatment of CLL). Though the following discussion relates to anti-CD20 antibodies of interest in treating B-cell lymphomas, the concepts are equally applicable to the foregoing list of antibodies.

As used herein, the term “anti-CD20 antibody” encompasses any antibody that specifically recognizes the CD20 B-cell surface antigen, including polyclonal anti-CD20 antibodies, monoclonal anti-CD20 antibodies, human anti-CD20 antibodies, humanized anti-CD20 antibodies, chimeric anti-CD20 antibodies, xenogeneic anti-CD20 antibodies, and fragments of these anti-CD20 antibodies that specifically recognize the CD20 B-cell surface antigen. Preferably the antibody is monoclonal in nature. By “monoclonal antibody” is intended an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site, i.e., the CD20 B-cell surface antigen in the present invention. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example.

Anti-CD20 antibodies of murine origin are suitable for use in the methods of the present invention. Examples of such murine anti-CD20 antibodies include, but are not limited to, the B1 antibody (described in U.S. Pat. No. 6,015,542); the 1F5 antibody (see Press et al. (1989) J. Clin. Oncol. 7:1027); NKI-B20 and BCA-B20 anti-CD20 antibodies (described in Hooijberg et al. (1995) Cancer Research 55:840-846); and IDEC-2B8 (available commercially from IDEC Pharmaceuticals Corp., San Diego, Calif.); the 2H7 antibody (described in Clark et al. (1985) Proc. Natl. Acad. Sci. USA 82:1766-1770; and others described in Clark et al. (1985) supra and Stashenko et al. (1980) J. Immunol. 125:1678-1685.

The term “anti-CD20 antibody” as used herein encompasses chimeric anti-CD20 antibodies. By “chimeric antibodies” is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to the CD20 cell surface antigen. The non-human source can be any vertebrate source that can be used to generate antibodies to a human CD20 cell surface antigen or material comprising a human CD20 cell surface antigen. Such non-human sources include, but are not limited to, rodents (e.g., rabbit, rat, mouse, etc.; see, for example, U.S. Pat. No. 4,816,567) and non-human primates (e.g., Old World Monkey, Ape, etc.; see, for example, U.S. Pat. Nos. 5,750,105 and 5,756,096). Most preferably, the non-human component (variable region) is derived from a murine source. As used herein, the phrase “immunologically active” when used in reference to chimeric anti-CD20 antibodies means a chimeric antibody that binds human C1q, mediates complement dependent lysis (“CDC”) of human B lymphoid cell lines, and lyses human target cells through antibody dependent cellular cytotoxicity (“ADCC”). Examples of chimeric anti-CD20 antibodies include, but are not limited to, IDEC-C2B8, available commercially under the name rituximab (Rituxan®;IDEC Pharmaceuticals Corp., San Diego, Calif.) and described in U.S. Pat. Nos. 5,736,137, 5,776,456, and 5,843,439; the chimeric antibodies described in U.S. Pat. No. 5,750,105; those described in U.S. Pat. Nos. 5,500,362; 5,677,180; 5,721,108; and 5,843,685.

Humanized anti-CD20 antibodies are also encompassed by the term anti-CD20 antibody as used herein. By “humanized” is intended forms of anti-CD20 antibodies that contain minimal sequence derived from non-human immunoglobulin sequences. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205. In some instances, framework residues of the human immunoglobulin are replaced by corresponding non-human residues (see, for example, U.S. Pat. Nos. 5,585,089; 5,693,761; 5,693,762). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al. (1986) Nature 331:522-525; Riechmann et al. (1988) Nature 332:323-329; and Presta (1992) Curr. Op. Struct. Biol. 2:593-596.

Also encompassed by the term anti-CD20 antibodies are xenogeneic or modified anti-CD20 antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598.

Fragments of the anti-CD20 antibodies are suitable for use in the methods of the invention so long as they retain the desired affinity of the full-length antibody. Thus, a fragment of an anti-CD20 antibody will retain the ability to bind to the CD20 B-cell surface antigen. Fragments of an antibody comprise a portion of a full-length antibody, generally the antigen binding or variable region thereof. Examples of antibody fragments include, but are not limited to, Fab, Fab′, F(ab′)₂, and Fv fragments and single-chain antibody molecules. By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778; 5,260,203; 5,455,030; 5,856,456. Generally, the Fv polypeptide further comprises a polypeptide linker between the V_{H} and V_{L} domains that enables the sFv to form the desired structure for antigen binding. For a review of sFv see Pluckthun (1994) in The Pharmacology of Monoclonal Antibodies, Vol. 113, ed. Rosenburg and Moore (Springer-Verlag, New York), pp. 269-315.

Antibodies or antibody fragments can be isolated from antibody phage libraries generated using the techniques described in McCafferty et al. (1990) Nature 348:552-554 (1990). Clackson et al. (1991) Nature 352:624-628 and Marks et al. (1991) J. Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

A humanized antibody has one or more amino acid residues introduced into it from a source that is non-human. These non-human amino acid residues are often referred to as “donor” residues, which are typically taken from a “donor” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting rodent CDRs or CDR sequences for the corresponding sequences of a human antibody. See, for example, U.S. Pat. Nos. 5,225,539; 5,585,089; 5,693,761; 5,693,762; 5,859,205. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

Further, any of the previously described anti-CD20 antibodies may be conjugated prior to use in the methods of the present invention. Such conjugated antibodies are available in the art. Thus, the anti-CD20 antibody may be labeled using an indirect labeling or indirect labeling approach. By “indirect labeling” or “indirect labeling approach” is intended that a chelating agent is covalently attached to an antibody and at least one radionuclide is inserted into the chelating agent. See, for example, the chelating agents and radionuclides described in Srivagtava and Mease (1991) Nucl. Med. Bio. 18: 589-603. Alternatively, the anti-CD20 antibody may be labeled using “direct labeling” or a “direct labeling approach”, where a radionuclide is covalently attached directly to an antibody (typically via an amino acid residue). Preferred radionuclides are provided in Srivagtava and Mease (1991) supra. The indirect labeling approach is particularly preferred. See also, for example, labeled forms of anti-CD20 antibodies described in U.S. Pat. No. 6,015,542.

The anti-CD20 antibodies are typically provided by standard technique within a pharmaceutically acceptable buffer, for example, sterile saline, sterile buffered water, propylene glycol, combinations of the foregoing, etc. Methods for preparing parentally administerable agents are described in Remington's Pharmaceutical Sciences (18^{th} ed.; Mack Pub. Co.: Eaton, Pa., 1990). See also, for example, International Publication No. WO 98/56418, which describes stabilized antibody pharmaceutical formulations suitable for use in the methods of the present invention.

The present invention also provides kits for use in the diagnostic methods of the invention. Such kits comprises at least one probe or primer that specifically hybridizes adjacent to or at a polymorphic region of the Fc gamma receptor IIIA (FcγRIIA) gene, where the polymorphic region comprises nucleotides encoding the FcγRIIIA 158F allele. Such a kit allows for detecting the presence of this allele in an individual, preferably detection of the homozygous 158F/F genotype. Alternatively, the kit comprises at least one probe or primer that specifically hybridizes adjacent to or at a polymorphic region of the Fc gamma receptor IIA (FcγRIIA) gene, where the polymorphic region comprises nucleotides encoding the FcγRIIA 131R allele. Such a kit allows for detecting the presence of at least one copy this allele in an individual. These kits can be combined, so that primers or probes specific to both genes are included in the kit. Further, the kits can comprise instructions for use.

The following examples are offered by way of illustration and not by way of limitation.

### EXPERIMENTAL

### EXAMPLE 1

### Materials and Methods

A. IL-2

The IL-2 formulation used is manufactured by Chiron Corporation of Emeryville, Calif., under the tradename Proleukin®. The IL-2 in this formulation is a recombinantly produced, unglycosylated human IL-2 mutein, called aldesleukin, which differs from the native human IL-2 amino acid sequence in having the initial alanine residue eliminated and the cysteine residue at position 125 replaced by a serine residue (referred to as des-alanyl-1, serine-125 human interleukin-2). This IL-2 mutein is expressed in E. coli, and subsequently purified by diafiltration and cation exchange chromatography as described in U.S. Pat. No. 4,931,543. The IL-2 formulation marketed as Proleukin® is supplied as a sterile, white to off-white preservative-free lyophilized powder in vials containing 1.3 mg of protein (22 MIU).

B. Anti-CD20 Antibody

The anti-CD20 antibody used in this and the following examples is Rituxan® (rituximab; IDEC-C2B8; IDEC Pharmaceuticals Corp., San Diego, Calif.). It is administered per its package insert dose (375 mg/m² infused over 6 hours).

C. Genotyping

Antibody-dependent cellular cytotoxicity (ADCC) mediated via IgG FcγR interaction with activating FcγR appears to be an important mechanism underlying the therapeutic activity of rituximab. Genetic polymorphisms in FcγRIIIA (CD16) and FcγRIIA (CD32) have been reported to influence the clinical response to rituximab in follicular lymphoma (FL) patients. See, e.g., Weng et al. (2003) J Clin Oncol. 21(21):3940-7. Interleukin-2 (Proleukin®) can induce expansion and activation of FcR bearing cells including natural killer (NK) cells, monocytes/ macrophages and neutrophils thereby augmenting ADCC mediated by monoclonal antibodies.

Thus, subjects were evaluated to determine their genotype for one or more known polymorphisms in FcγRIIIa, including the bi-allelic functional polymorphism (G→T) at nucleotide position 559, which predicts a valine (V) to phenylalanine (F) substitution at amino acid position 158, in order to determine their FcγRIIIa genotype at this position (158 FF, 158 FV, or 158 VV). See, e.g., Koene et al. (1997) Blood 90(3):1109-14. The subject's genotype at one or more additional polymorphisms (e.g., 48 L/R/H, 131 R/H, 176 F/V, etc.) can also be determined. See, e.g., Weng et al. (2003), supra; de Vries et al. (1996) Blood 88(8):3022-7; de Haas et al. (1996) J Immunol. 1996 156(8):3948-55.

Genotyping of subjects was conducted essentially as described in Koene et al. (1997) Blood 90:1109-1114 and/or Leppers-van de Straat et al. (2000) J Immunol Methods 242(1-2): 127-32. Briefly, polymerase chain reaction (PCR) was used to amplify a sequence containing the target polymorphism from a sample (e.g., whole blood or PBMCs) obtained from the subject to be genotyped. Alternative methods for genotyping are described in detail in U.S. Provisional Application Ser. No. 60/560,649, filed Apr. 7, 2004, which application is hereby incorporated by reference in its entirety herein.

D. Grading of Response

Grading of tumor response is based upon the report of the International Workshop to Standardize Response Criteria for Non-Hodgkin's Lymphomas (see, Cheson et al. (1999) J. Clin. Oncol. 17:1244-1253) and protocol-defined criteria as follows:
Complete response (CR)—Defined as absence of clinically detectable disease with normalization of any previously abnormal radiographic studies, bone marrow and cerebrospinal fluid (CSF). Response must persist for at least one month. Patients with bone marrow positive for lymphoma prior to chemotherapy must have a repeat biopsy, which is confirmed after a month, negative for lymphoma.
Partial response (PR)—Defined as at least 50% decrease in all measurable tumor burden in the absence of new lesions and persisting for at least one month (applicable to measurable tumors only).

Patients were also assessed (e.g., for effects of Proleukin® IL-2 and rituximab therapy) on the following:
Response duration—Defined as the time from first documented response until progressive disease.
Time to progression—Defined as the time from study entry to progressive disease, relapse or death.
Stable disease (SD)—Defined as a less than 50% reduction in tumor burden in the absence of progressive disease.
Progressive disease (PD)—Defined as representing 25% or greater increase in tumor burden or the appearance of a new site of the disease.

Relapse (R)—Defined as the appearance of tumor following documentation of a complete response.

### EXAMPLE 2

### Combination IL2-Rituximab in Xenograft Models of Human B-Cell Non-Hodgkin's Lymphoma

Combination IL-2 (Proleukin®) and Rituximab administration was evaluated in two distinct xenograft models of human B-cell lymphoma as follows. See, e.g., Hudson et al. (1998) Leukemia 12(12):2029-2033 for a description of Namalwa and Daudi xenograft models.

Namalwa and Daudi human B-cell lines were grown as subcutaneous tumors (staged at 100-200 mm³) in NK-competent Balb/c nude mice (n=10/group). The Namalwa/Balb/c nude mouse model is associated with low level CD20 expression and is regarded as a model of aggressive/high grade disease. The Daudi/Balb/c nude model expresses high levels of CD20 and is associated with a less aggressive/low grade disease profile. Furthermore, NK cells cannot lyse Daudi tumor cells in the absence of activation by cytokines such as IL-2. See, e.g., Damle et al. (1987) J. Immunol. 138(6):1779-1785. Selected characteristics of the different mouse models are shown below:

| Characteristic | Namalwa | Daudi |
|---|---|---|
| CD20 expression | Low | High |
| Disease Status | Aggressive | Low Grade |
| Rituximab efficacy | Resistant | Responsive |
| IL-2 efficacy | Effective | Low efficacy |
| Model Duration | 2 weeks | 6 weeks |

Namalwa or Daudi tumor cells were implanted into the mice and rituximab and/or IL-2 administration began when the tumors were staged at staged at 100-200 mm³, typically 8-12 days following tumor cell implantation.

Single-agent dosage regimes were as follows. One group of mice received daily subcutaneous (s.c.) IL-2 at 0.25 mg/kg (low dose daily group). Another group of mice received thrice-weekly IL-2 at (1 mg/kg, s.c.), on days 1, 3, 5, 8, 10, 12, 15, 17, 19, 22, 24 and 26. A third group of mice received i.v. or i.p. Rituximab on days 1, 8, 15 and 22 (e.g., 10 mg/kg, 1x/wk, i.p.). Furthermore, in the Daudi mice, an additional group received i.v. or i.p. rituximab F(ab′)₂ fragment Ix/week (days 1, 8, 15, and 22) at 10 mg/kg. Control animals received vehicle only.

Combination-agent dosage regimes were also tested by administering rituximab on days 1, 8, 15, and 22 to animals receiving either the daily or thrice-weekly administration of IL-2, at dosages described above. A group of Daudi animals also received a combination of IL-2 (daily or thrice-weekly) and rituximab F(ab′) (1x/week). All single agent and combination agent dosage regimes were well tolerated.

A. Namalwa Model

In the Namalwa mouse model, daily or thrice weekly administration of IL-2 as a single-agent were equally effective in inhibiting tumor growth. In particular, daily and thrice-weekly IL-2 dosage regimes resulted in statistically significant inhibition of between about 40-60% tumor growth, p<0.05, ANOVA) tumor growth in the Namalwa mouse model.

Namalwa tumors were generally resistant to rituximab. No difference in tumor efficacy when rituximab administered at 10, 25 or 50 mg/kg, 1x/wk (˜0-30% tumor growth inhibition, p>0.05, ANOVA) was seen in Namalwa animals.

Namalwa tumor animals receiving combination rituximab-IL-2 administration showed a marginally higher efficacy with daily IL-2 (0.5 mg/kg, s.c.) in combination with once weekly rituximab (10 mg/kg) as compared to animals receiving IL-2 alone (p=0.046, ANOVA). Furthermore, combination thrice-weekly IL-2 (1 mg/kg) with rituximab 10 mg/kg showed no improvement over animals receiving IL-2 alone (1 mg/kg, 3x/wk, p>0.05, ANOVA).

B. Daudi Model

Daudi tumor animals were typically resistant to single-agent IL-2 administration (either daily or thrice weekly). Daudi tumor volume in animals receiving daily IL-2 alone (0.5 mg/kg, daily×12 followed by 1 wk off for 2 cycles) was slightly reduced as compared to controls (p =0.047, ANOVA). Thrice-weekly administration of IL-2 (1 or 1.5 mg/kg 3x/wk×4 wk) to Daudi tumor animals also exhibited slightly reduced tumor volume as compared to controls (p=0.01, ANOVA).

However, Daudi tumors were highly responsive to rituximab administration. Significant growth inhibition of Daudi tumors and dose-response effects were seen in animals receiving 10 and 50 mg/kg, 1x/wk rituximab. Similar results were obtained using combination rituximab (10 mg/kg, 1x/wk) and daily IL-2 (0.25 mg/kg, daily) administration.

Strikingly, combined administration of thrice-weekly IL-2 and once-weekly rituximab resulted in significant tumor growth inhibition and objective tumor responses as compared to single agent IL-2, single agent rituximab and combination daily IL-2 and weekly rituximab. The clear synergy between IL-2 and rituximab was also evidenced by a significant delay in time to progression by 41 days and 57 days compared to Rituximab and IL-2, respectively.

Furthermore, no significant difference was observed between single agent IL-2 administration and combined rituximab F(ab′)₂ 10 mg/kg and thrice weekly IL-2 (1 mg/kg, 3x/wk) administration, indicating that the efficacy of IL-2 and rituximab combination therapy is dependent upon IgG1 Fc-mediated effector mechanisms in the Daudi model.

Thus, the combination of IL-2 and rituximab in the Daudi xenograft model results in significant and durable tumor responses. Clinical observations are summarized in Table A.

TABLE A

| | Tumor responses 2 | | |
|---|---|---|---|
| Treatment to | (CR/PR/MR + SD)* | | Median time |
| (n = 20 mice/gp, | End of treatment | End of study | to 1000 mm³ |
| 2 independent studies) | cycle (day 30) | (˜Day 80) | (days) |
| Vehicle | 0 | 0 | 17 |
| thrice-weekly IL-2 | 0 CR | 1 CR | 21 |
| (1 mg/kg) | | | |
| rituximab (10 mg/kg, | 1 PR, 5 MR + SD | 3 CR, 1 PR, | 42 |
| 1x/wk) | | 3 MR + SD | |
| Thrice weekly IL-2 | 4 CR, 4 PR, | 4 CR, 1 PR, | >85 |
| (1 mg/kg) + rituximab | 9 MR + SD | 7 MR + SD | |
| (10 mg/kg, 1x/wk) | | | |

*Responses were defined by degree of regression from initial, i.e., CR (100%); PR (50-99%); MR (25-49%); SD (±25%)

In sum, single agent IL-2 was more effective in aggressive/high grade Namalwa model compared to the less aggressive/low grade Daudi model. Furthermore, tumor responsiveness to rituximab correlated well with phenotypic CD20 expression, i.e., Daudi CD20high>Namalwa CD20low) and appeared to inversely relate to disease status (low grade Daudi>high grade Namalwa). In the high grade Namalwa model, daily administration of IL-2 and rituximab exhibited marginally incremental efficacy compared to single agent IL-2. In the Daudi model, thrice weekly IL-2 and rituximab clearly demonstrated synergistic effects and increased time to progression in the low grade Daudi tumor model. An F(ab′)2 fragment of rituximab abrogated activity, revealing the critical role of IgG1 Fc-FcR mediated ADCC in the augmentation of anti-tumor responses by IL-2/rituxan combination therapy.

### EXAMPLE 3

### Phase I Combination IL2-Rituximab Therapy

Two parallel Phase I studies were conducted to evaluate combination therapy with rituximab and IL-2 in relapsed or refractory B-cell non-Hodgkin's lymphoma (NHL) patients. See, Gluck et al. (2004) Clin Cancer Res. 10(7):2253-2264.

Thirty-four patients with advanced NHL received rituximab (375 mg/m(2) i.v. weekly, weeks 1-4) and escalating doses of s.c. IL-2 (2-7.5 million international units (MIU) daily (n=19), e.g., 2, 4.5, 6, and 7.5 MIU) or 4.5-18 MIU (e.g., 4.5, 10, 14 or 18 MIU) three times weekly (n=15), weeks 2-5).

The maximum tolerated dose of IL-2 determined from these studies was either 6 MIU daily s.c. IL-2 or 14 MIU thrice/weekly.

Of the 9 patients enrolled at the daily schedule MTD, 5 showed clinical response. On the thrice-weekly schedule at the MTD, 4 of 5 patients responded and had greater increases in NK cell counts than daily dosing. Responses were seen in various NHL subtypes. In subjects receiving daily IL-2, responses were seen with diffuse large cell, MALT, follicular, and lymphoplasmacytic lymphomas. In subjects receiving thrice-weekly IL-2, responses were seen with diffuse large cell, follicular, small cell, follicular center, follicular mixed, marginal zone, and mantle cell lymphoma patients. All responses appeared to be durable.

The number of NK cells correlated with clinical response on the thrice-weekly regimen. At the maximum dose levels, median NK cell counts were highest at week 5. In addition, ADCC activity was increased and maintained after IL-2 therapy in responding and stable disease patients. See, also Gluck et al. (2004) Clin Cancer Res. 10(7):2253-2264.

Thus, addition of IL-2 to rituximab therapy is safe and, using thrice-weekly IL-2 dosing, results in NK cell expansion that correlates with response.

### EXAMPLE 4

### Combination IL2-Rituximab Therapy in Rituximab-Refractory or Relapsed Subjects

A phase II trial (denoted IL2NHL03 herein) evaluating the combination of IL-2 (Proleukin®) and rituximab in low grade/follicular non-Hodgkin lymphoma (NHL) patients who were rituximab refractory or relapsed within 6 months of rituximab treatment has been initiated. Rituximab was administered weekly at weeks 1, 2, 3 and 4 at a dose of 375 mg/m² (IV) and Proleukin® was given subcutaneously (SC) three times weekly for eight weeks (14 MIU during weeks 2, 3, 4 and 5; 10 MIU during weeks 6, 7, 8 and 9). Endpoints of this study have included overall response rate (ORR), NK cell expansion and evaluation of NK cell function and FcγRIIIA and FcγRIIA polymorphisms.
An evaluable patient was defined as: subjects must have received 4 weeks of rituximab therapy and 70% of the prescribed Proleukin® dose and schedule. The response was evaluated as follows. Tumor measurements were based upon measurements of perpendicular diameters, using the longest diameter and its greatest perpendicular.

Forty-four patients have been enrolled to date and 27 are currently evaluable for tumor response at week 16. Five clinical responses have been documented, with two complete and three partial responses including a PR in a patient who had failed prior Y-90 ibritumomab tiuxetan (Zevalin); 5 patients had stable disease.

A. 158 F/V Polymorphism

Twenty patients were genotyped, as described above in Example 1. In this group of 20, 4 clinical responses have been documented, including one complete and 3 partial recoveries. (Table B, below). In addition, 4 patients had stable disease (SD) lasting 4 or more months.

TABLE B

| | | | | Duration |
|---|---|---|---|---|
| ID | Sex/Race | Histology | Response | (months) |
| 12011 | M/C | Follicular Grade II | CR | 8.5 |
| 01001 | F/C | Extranodal MZL | PR | 9.7 |
| 19004 | F/C | Follicular Grade I | PR | 9.2 |
| 17001 | M/C | Follicular Grade II | PR | 1.9 |

Notably, the frequency of the FcγRIIIA 158 allotypes in this rituximab refractory/relapsed population were significantly skewed with a marked increase in homozygous 158 F/F (13/20; 65%) subjects and a decreased frequency of heterozygous FcγRIIIA 158 V/F (5/20; 25%) compared to 32-39% and 46-51% in normal/reported FL NHL populations respectively. See Table 1 below.

TABLE 1
Higher frequency of FcγRIIIA 158F/F polymorphism in IL2NHL03 study
patient population.

| | | | FcγRIIIA Polymorphism | | | | | |
|---|---|---|---|---|---|---|---|---|
| Study | Population | # Subjects | 158VV | | 158VF | | 158FF | |
| Koene | Normal | 87 | 15 | 17% | 44 | 51% | 28 | 32% |
| | Caucasian | | | | | | | |
| Cartron | Previously | 55 | 10 | 20% | 22 | 45% | 17 | 35% |
| | unTx follicular | | | | | | | |
| | CD20 + NHL | | | | | | | |
| Weng & Levy | Previously | 87 | 13 | 15% | 40 | 46% | 34 | 39% |
| | Tx(Rituxan/Chemo) | | | | | | | |
| | follicular NHL | | | | | | | |
| IL2NHL03 | Rituxan | 20 | 2 | 10% | 5 | 25% | 13 | 65% |
| | relapsed/refractory | | | | | | | |
| | indolent NHL | | | | | | | |

Strikingly, the clinical responders that have been genotyped for the FcγRIIa 158 polymorphism all expressed the FcγRIIIA 158F/F genotype, which is associated with poorer response rates and duration of response to rituximab alone. See Table 2 below.

TABLE 2
Association of FcγRIIIA 158V/F polymorphism and clinical
response profile in IL2NHL03 study patient population.

| | | FcγRIIIA Genotype | | | | | | | |
|---|---|---|---|---|---|---|---|---|---|
| Study | Objective | 158VV | | 158VF | | 158FF | | 158F Carrier | |
| Cartron | M2 | 10/10 | 100% | | | | | 26/39 | 67% |
| | M12 | 9/10 | 90% | | | | | 20/39 | 51% |
| Weng | M1-3 | 12/13 | 92% | 21/40 | 53% | 23/34 | 68% | 44/74 | 59% |
| | M12 | 9/12 | 75% | 8/35 | 23% | 8/27 | 30% | 16/62 | 26% |
| IL2NHL03 | M1-4 | 0/2 | 0% | 0/5 | 0% | 4/13 | 31% | 4/18 | 22% |

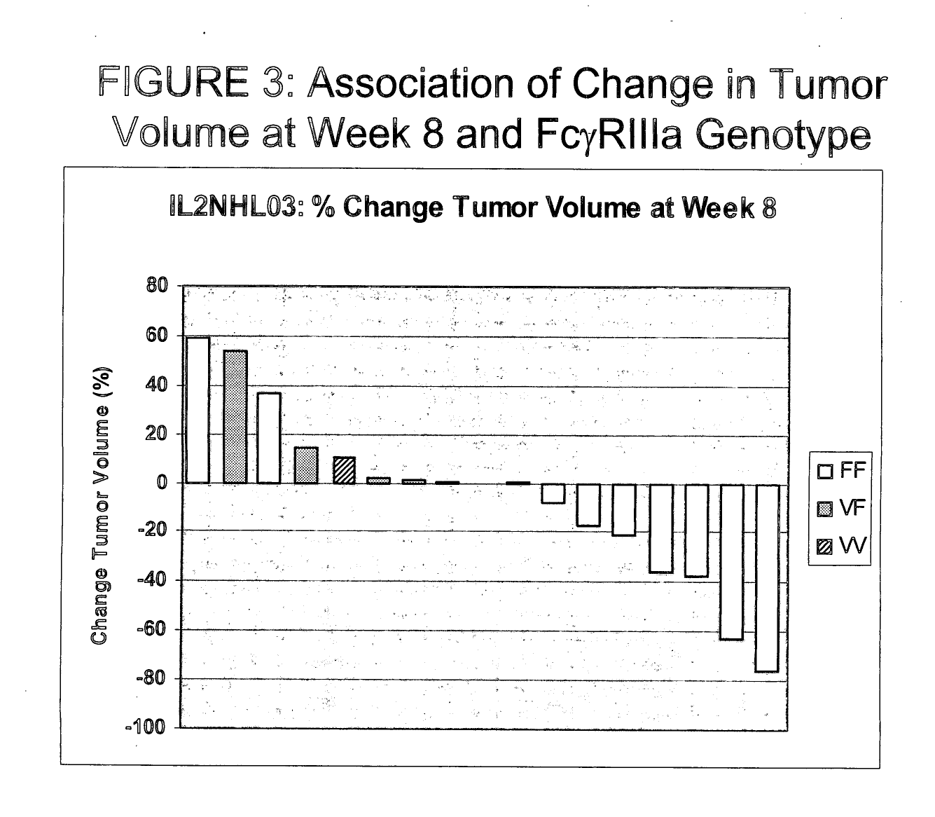

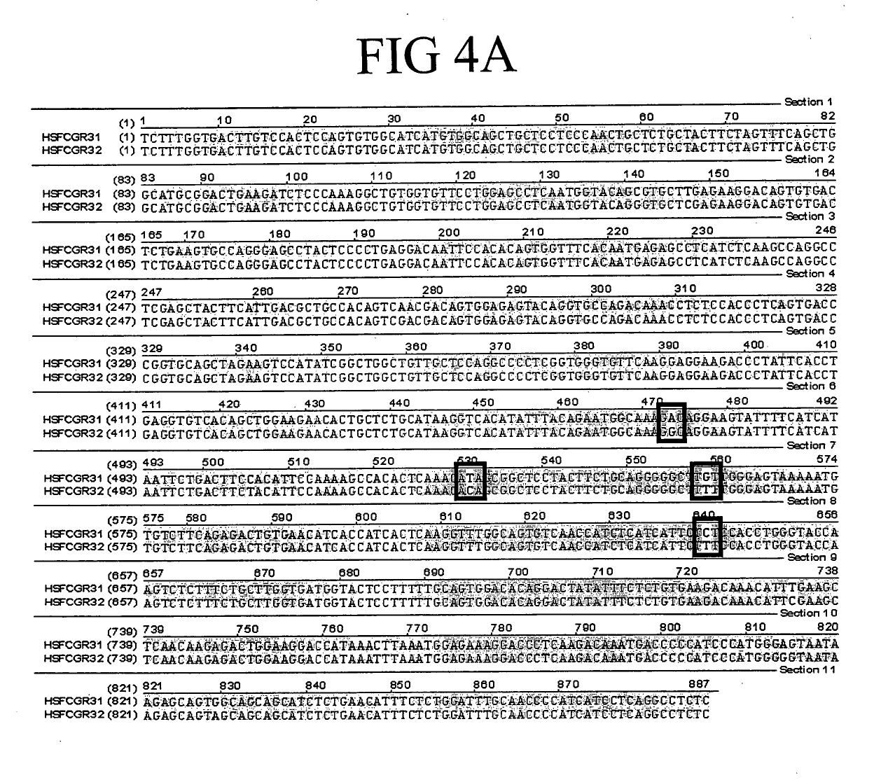

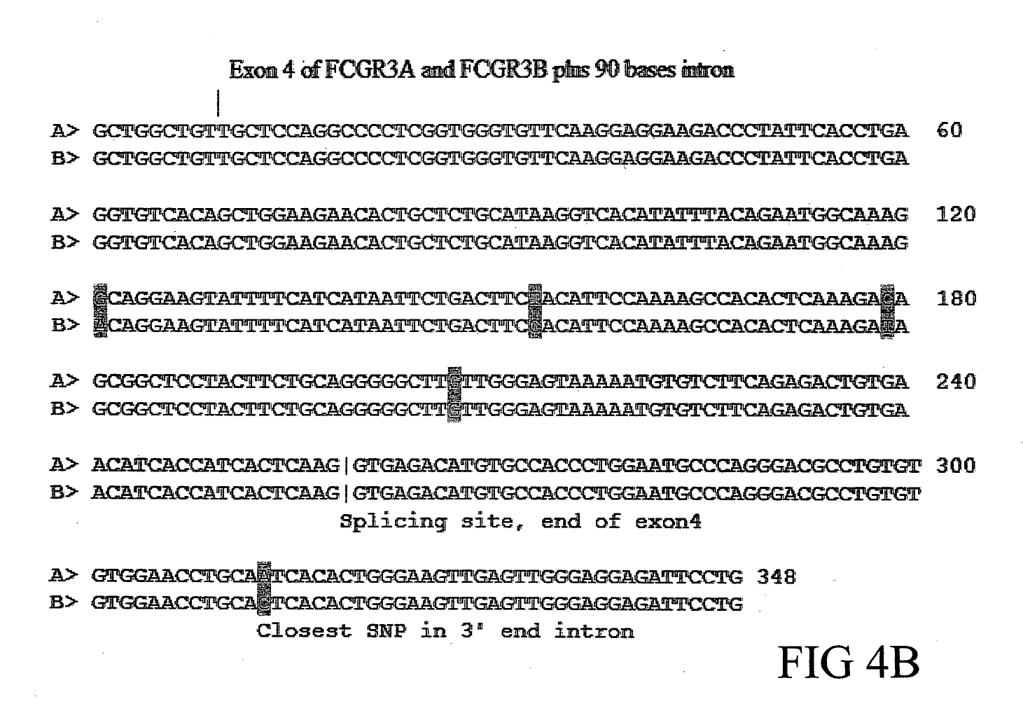

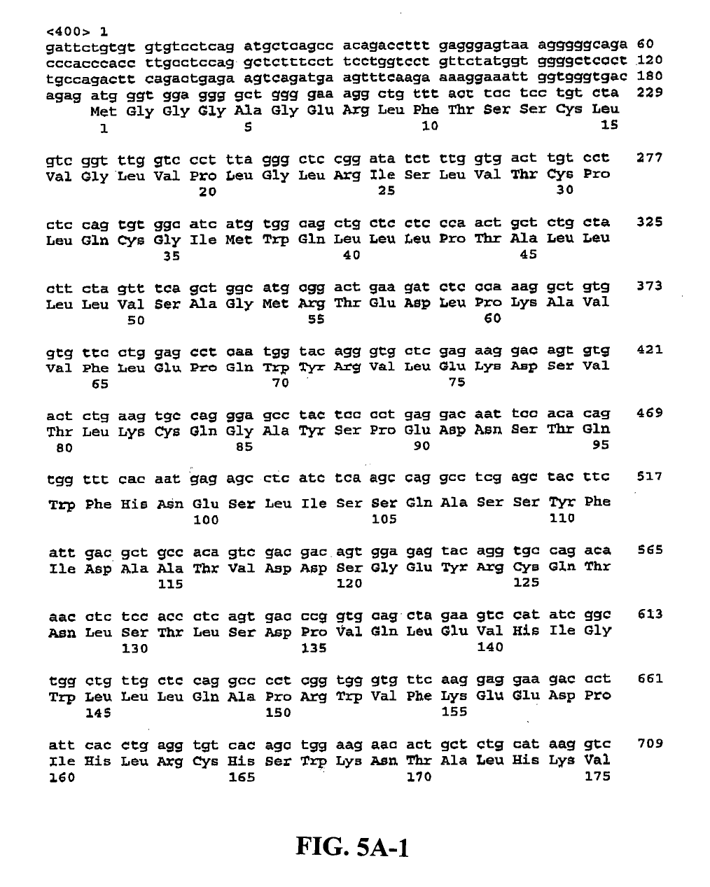

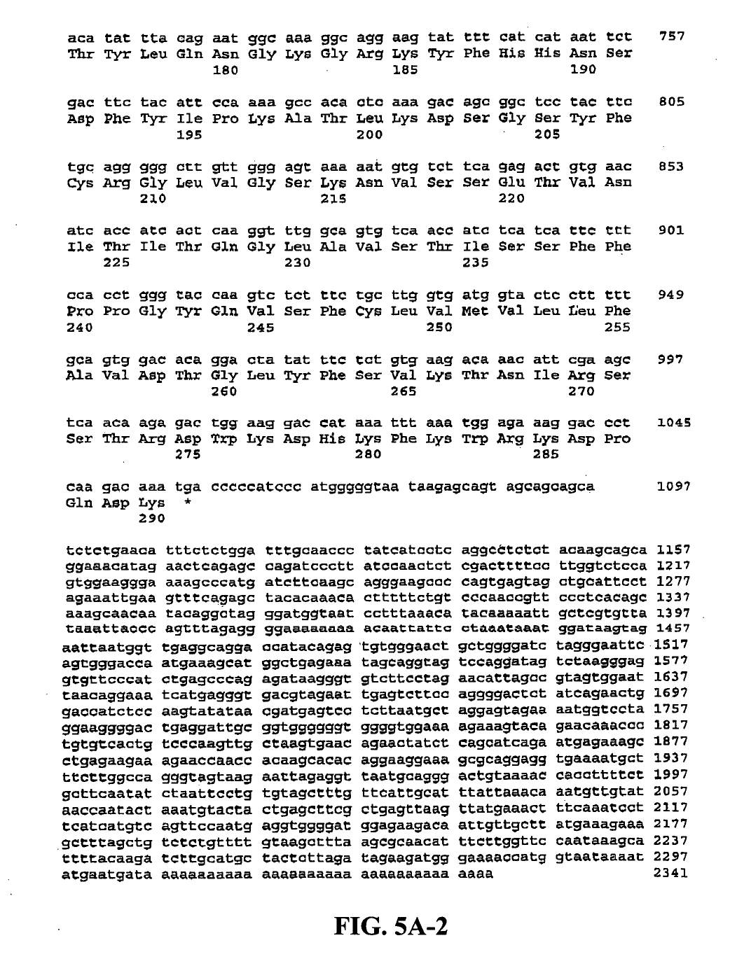

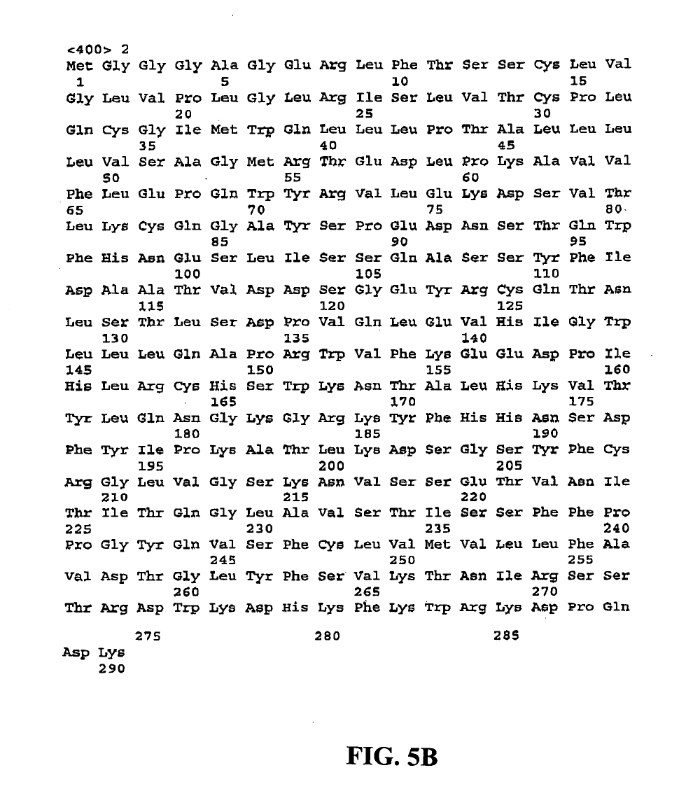

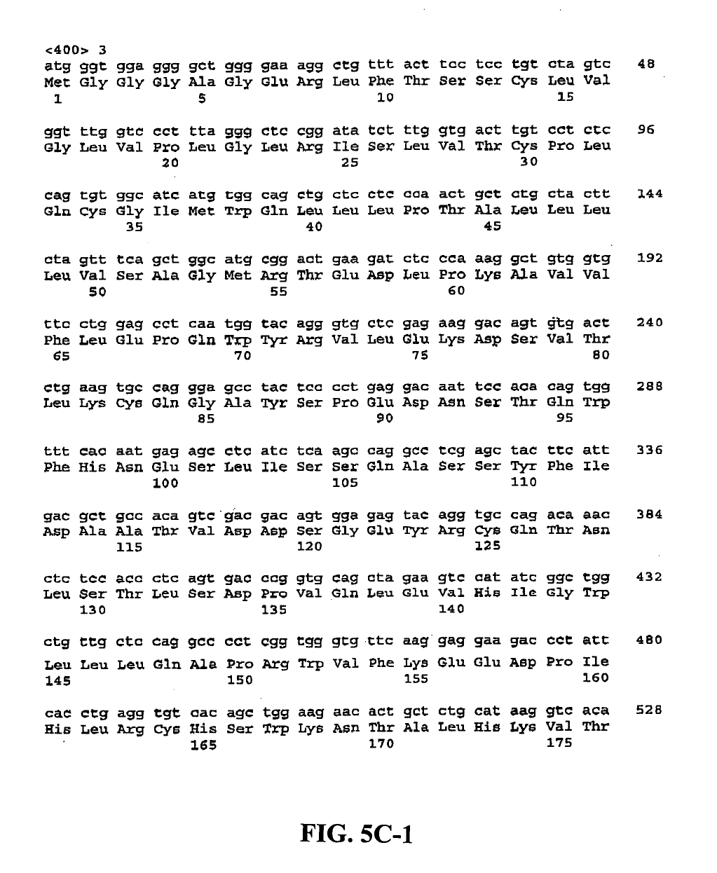

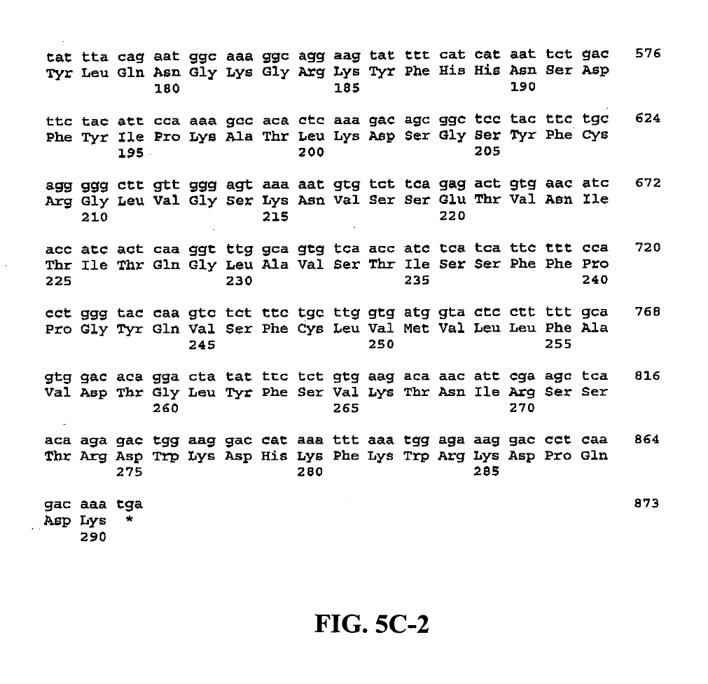

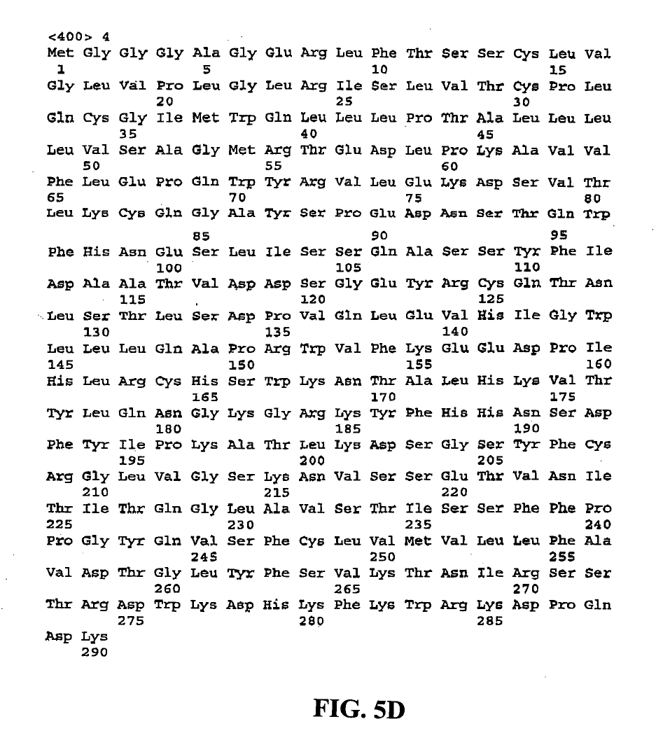

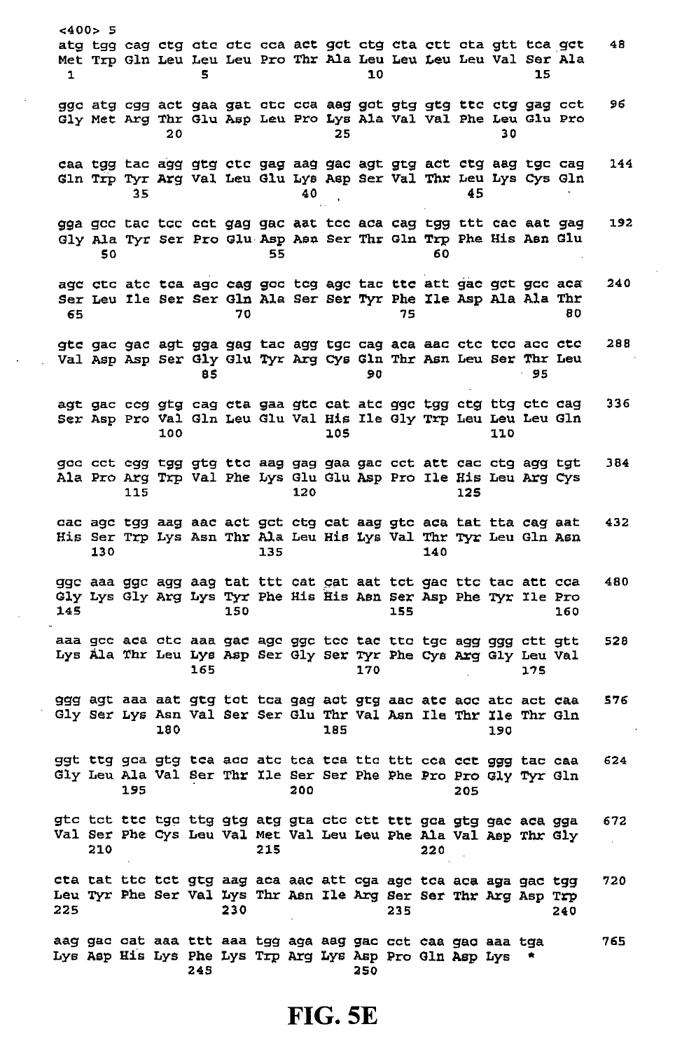

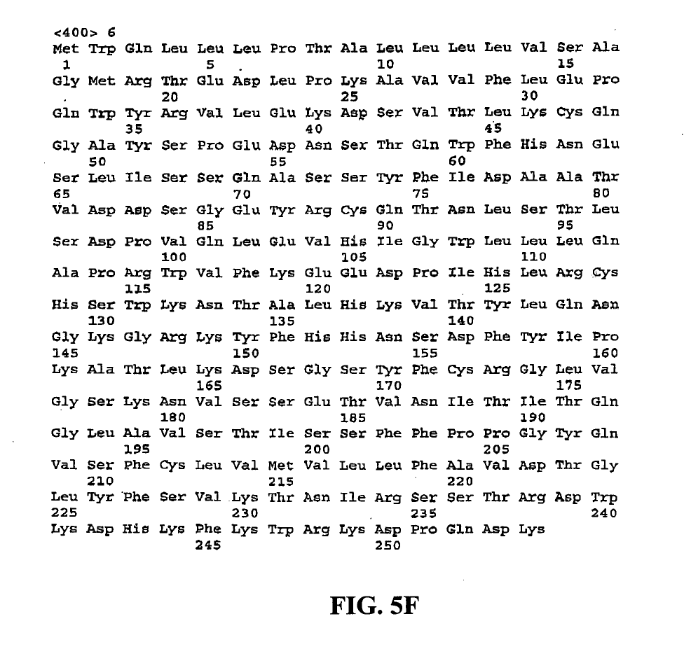

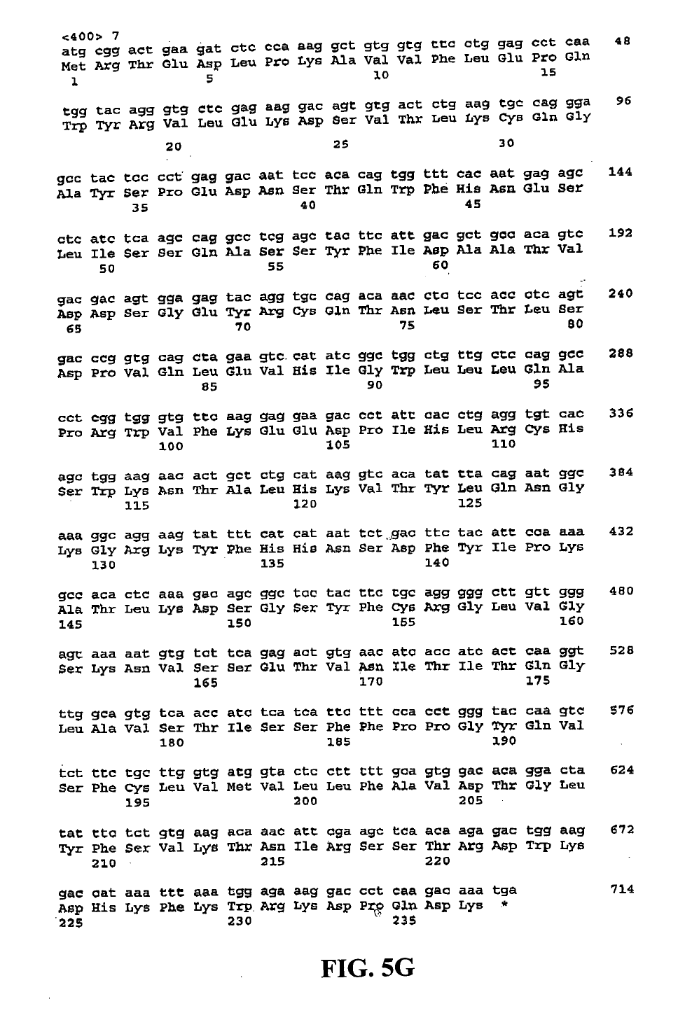

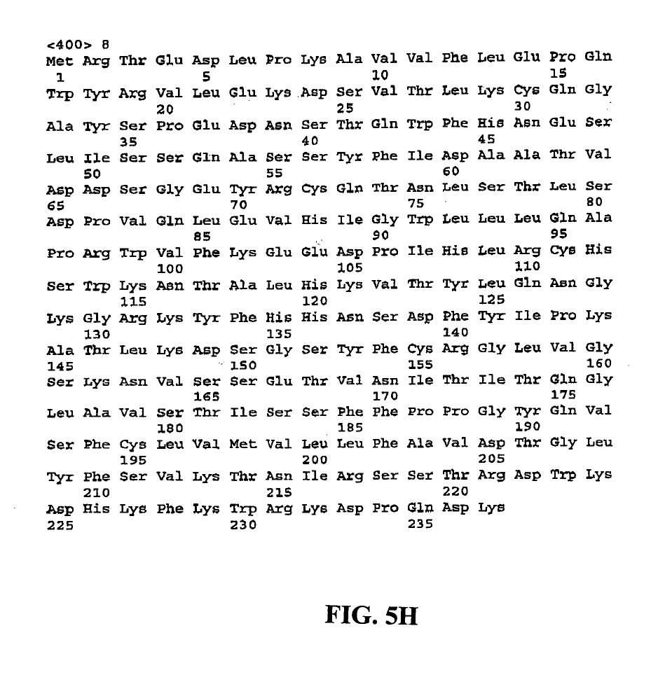

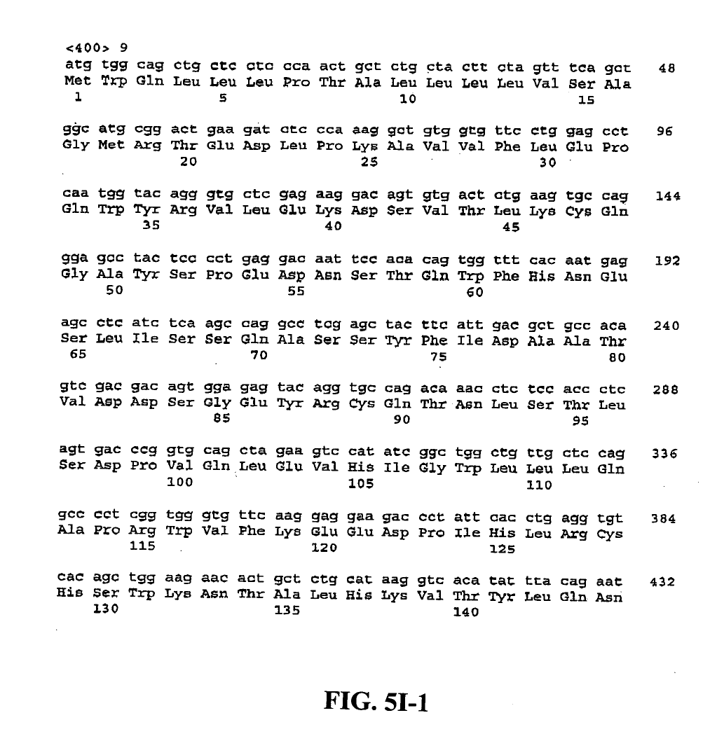

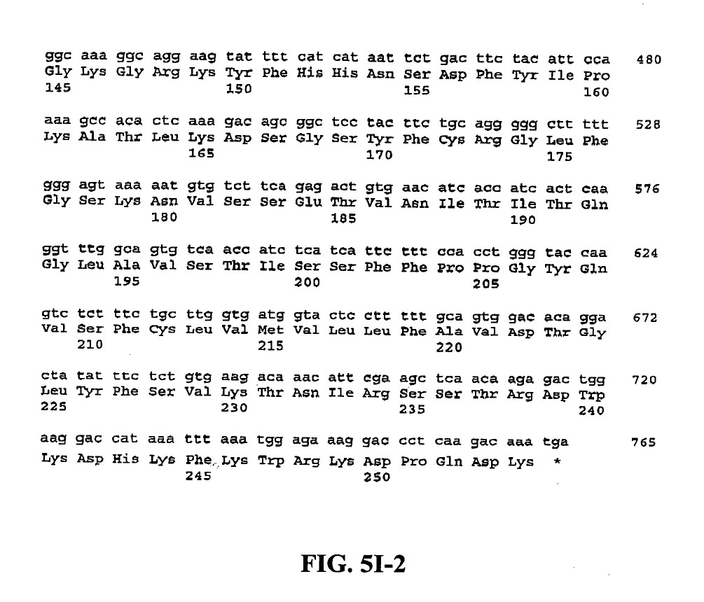

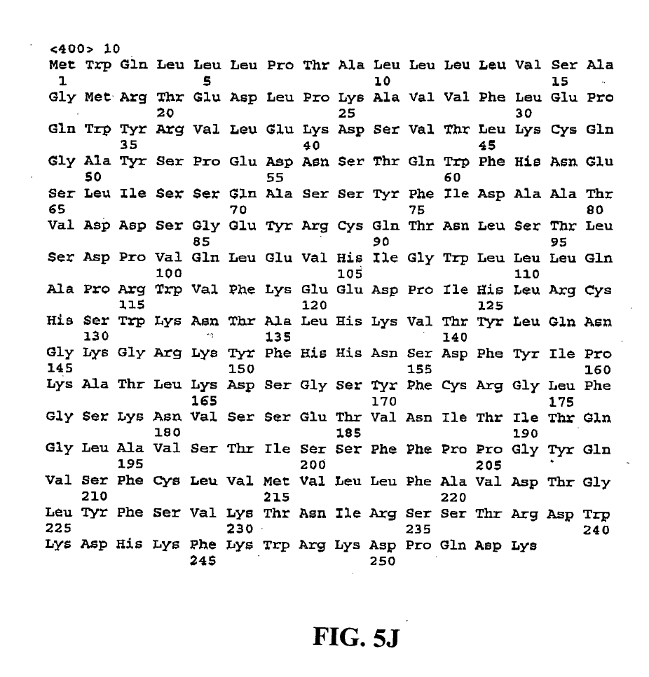

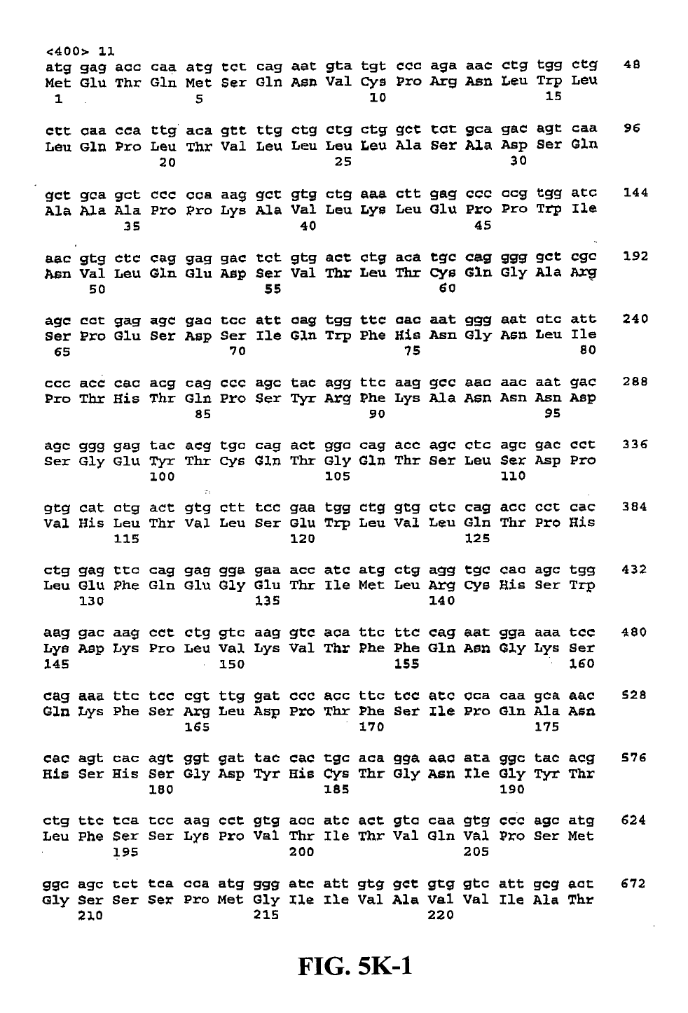

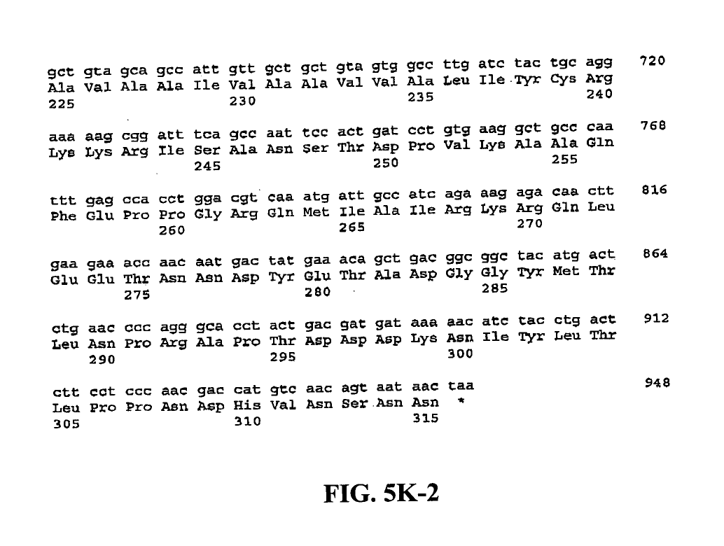

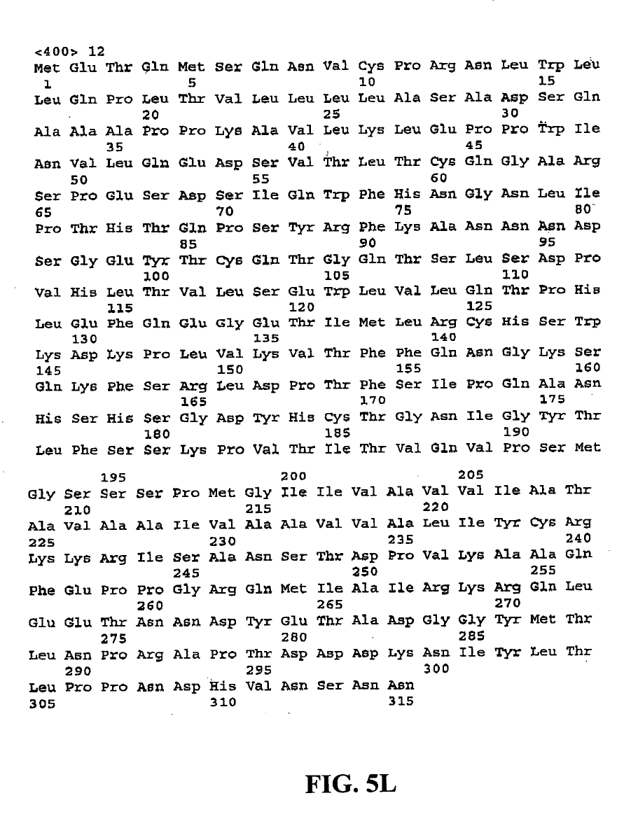

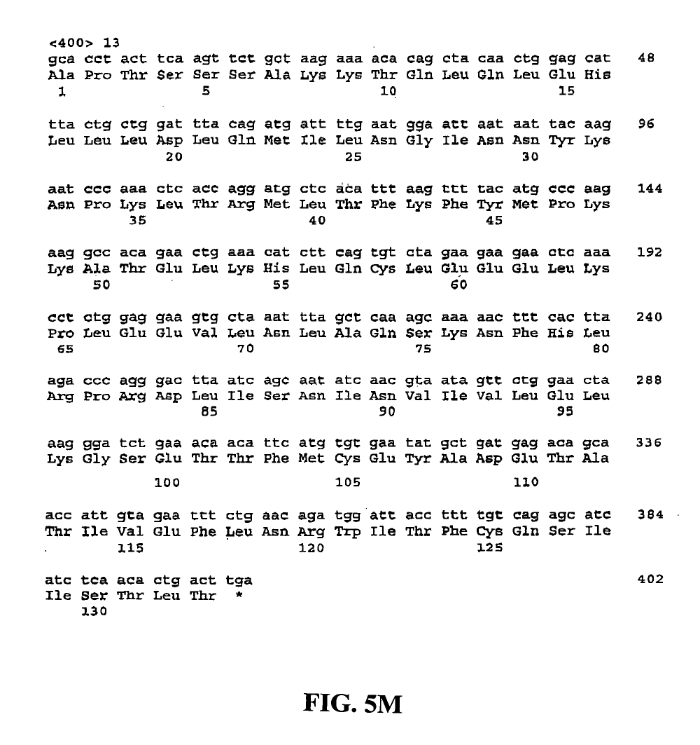

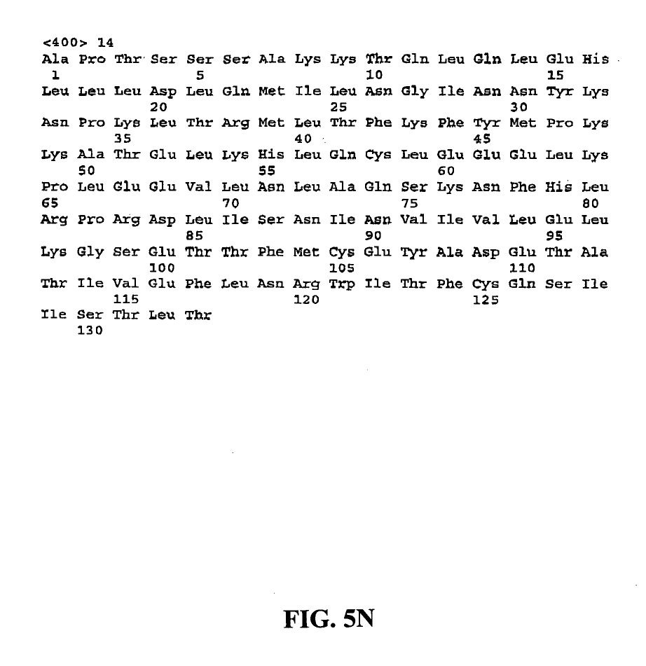

Furthermore, when the percent change in tumor volume was measured in genotyped patients, tumor volume shrunk significantly more in 158F/F patients. (FIG. 3).

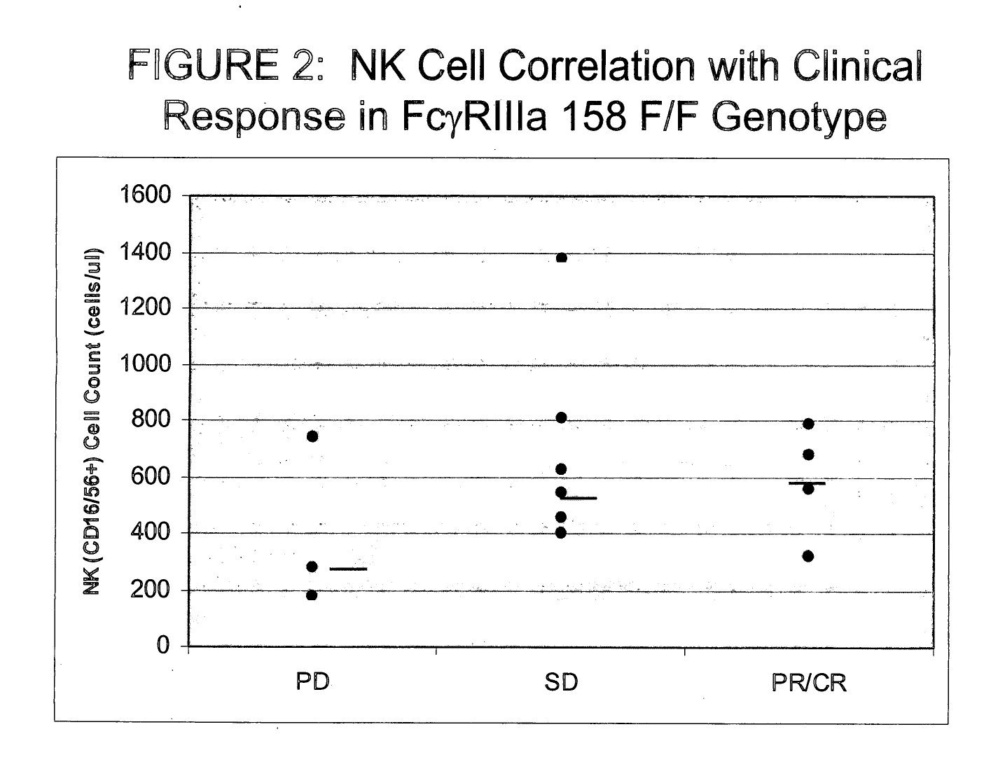

NK cell counts were obtained in the subset of patients that were FcγRIIIA 158F/F carriers at week 10 of the study, and correlated with clinical status. Results are shown in FIG. 2. These data show a positive correlation of NK CD16+CD56+ cell number with disease status (PD=progressive disease; SD=stable disease; PR/CR=partial/complete response).

Table 3 summarizes the relationship between low-grade NHL disease types and FCγRIIIA 158 genotypes in this study.

TABLE 3
Relationship between Low Grade NHL Disease Types
and FcγRIIIA 158 Genotypes

| FcγRIIIA 158 V/V | | FcγRIIIA 158 V/F | | FcγRIIIA 158 F/F | |
|---|---|---|---|---|---|
| FLC | SD | EA: SLL/CLL | SD | FMC | CR |
| Plasmacytoid | PD | Xnodal, MZL | SD | FMC | PR |
| SLL | PD | Follicular-gr | PD | Xnodal MZL | PR |
| | | | | FSC | PR |
| | | | | FMC | SD |
| | | | | FMC/diffuse | SD |
| | | | | MALT | SD |
| | | | | MZL Splenic | SD |
| | | | | MZL | SD |
| | | | | Follicular | PD |
| | | | | SLL | PD |

B. 131 H/R Polymorphism

The genotypes of the rituximab refractory or relapsed patients also exhibited increased an increase in the proportion of homozygous FcγRIIA 131H/H patients (7/17 (42%) and a decrease in FcγRIIA 131 H/R patients (5/17 (29%) as compared to other patient populations. See Table 4 below.

TABLE 4
Higher frequency of FcγRIIA 131H/R polymorphism in
IL2NHL03 study patient population.

| | | # | FcγRIIA Polymorphism | | | | | |
|---|---|---|---|---|---|---|---|---|
| Study | Population | Subjects | 131HH | | 131HR | | 131RR | |
| Lehrnbecher | Normal | 2419 | 627 | 26% | 1223 | 50% | 579 | 24% |
| | Caucasian | | | | | | | |
| Weng & | Previously | 87 | 20 | 23% | 43 | 49% | 24 | 28% |
| Levy | Tx(Rituxan/Chemo) | | | | | | | |
| | follicular NHL | | | | | | | |
| IL2NHL03 | Rituxan | 17 | 5 | 29% | 5 | 29% | 7 | 42% |
| | relapsed/refractory | | | | | | | |
| | indolent NHL | | | | | | | |

Furthermore, all clinical responders evaluated to date are FcγRIIA 131-R carriers associated with poor outcome to rituximab therapy. See Table 5 below.

TABLE 5
Association of FcγRIIA 131 H/R polymorphism and clinical
response profile in IL2NHL03 study patient population.

| | | FcγRIIA Genotype | | | | | | | |
|---|---|---|---|---|---|---|---|---|---|
| Study | Objective | 131HH | | 131HR | | 131RR | | 131R Carrier | |
| Weng | M1-3 | 16/20 | 80% | 27/43 | 63% | 13/24 | 54% | 40/67 | 60% |
| | M12 | 11/20 | 55% | 10/37 | 27% | 4/17 | 24% | 14/54 | 26% |
| IL2NHL03 | M1-4 | 0/4 | 0% | 2/5 | 40% | 1/7 | 14% | 3/12 | 25% |

In conclusion, genetic polymorphisms FcγRIIIA 158F/F and FcγRIIA 131 R/R are associated with poor clinical response to single agent rituximab. However, immunotherapeutic intervention with IL-2, which effectively expands and activates FcγR-bearing cells, may achieve a critical threshold of NK cell number sufficient to drive ADCC more effectively in patients carrying low affinity IgG FcγR allotypes and thus restoring the potential of such individuals to respond effectively to anti-cancer monoclonal antibody therapy.

### EXAMPLE 5

### Combination IL2-Rituximab Therapy in Naive Subjects

Rituximab naive subjects with follicular non-Hodgkin's lymphoma (NHL), refractory or relapsed after previous chemotherapy, are examined for the relationship between FcγRIIIA polymorphisms at amino acid positions 158 and 131 and clinical response to rituximab alone and in combination with IL-2. Treatment arms are stratified by polymorphism status, and subjects receive rituximab alone (i.v., 375 mg/m² weekly for 4 weeks), or rituximab according to this dosing protocol in combination with thrice-weekly, subcutaneous rhIL-2 (Proleukin®), for 8 weeks (14 MIU for first 4 weeks, followed by 10 MIU for 4 weeks).

Whole blood samples and tumor biopsies are collected for subsequent gene expression profiling, and characterization of the genotypes for these two FcγRIIIA polymorphisms and the FcγRIIA polymorphism. Clinical outcome at week 14 weeks post initiation of treatment protocols is correlated with genotype and NK cell count.

All publications and patent applications mentioned in the specification are indicative of the level of those skilled in the art to which this invention pertains. All publications and patent applications are herein incorporated by reference in their entireties to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the scope of embodiments disclosed herein.

What is claimed is:

1. A diagnostic method for predicting therapeutic response to interleukin-2 (IL-2) immunotherapy in an individual in need thereof, said method comprising detecting the allelic pattern for the Fc gamma receptor IIIA (FcγRIIIA) gene of said individual, wherein the presence of the homozygous FcγRIIIA 158F/F genotype is indicative of an individual that will exhibit a positive therapeutic response to said IL-2 immunotherapy.

2. The method of claim 1, wherein said individual is need of IL-2 immunotherapy for treatment of a cancer.

3. The method of claim 2, wherein said individual is also undergoing treatment with an antibody that targets a cell-surface antigen expressed on the surface of cells of said cancer.

4. The method of claim 3, wherein said antibody is an immunoglobulin G1 (IgG1) monoclonal antibody.

5. The method of claim 2, wherein said cancer is a B-cell lymphoma.

6. The method of claim 5, wherein said B-cell lymphoma is non-Hodgkin's B-cell lymphoma.

7. The method of claim 2, wherein said cancer is selected from the group consisting of breast cancer, ovarian cancer, cervical cancer, prostate cancer, colon cancer, melanoma, renal cell carcinoma, acute myeloid leukemia (AML); and chronic lymphocytic leukemia (CLL).

8. The method of any one of claim 1, wherein the allelic pattern for said FcγRIIIA gene is detected by a method selected from the group consisting of allele specific hybridization, primer specific extension, oligonucleotides ligation assay, restriction enzyme site analysis, and single-stranded conformation polymorphism analysis.

9. A diagnostic method for predicting therapeutic response to interleukin-2 (IL-2) immunotherapy in an individual in need thereof, said method comprising detecting the allelic pattern for the Fc gamma receptor IIA (FcγRIIA) gene of said individual, wherein the presence of the heterozygous FcγRIIA 131H/R genotype or the presence of the homozygous FcγRIIA 131R/R genotype is indicative of an individual that will exhibit a positive therapeutic response to said IL-2 immunotherapy.

10. The method of claim 9, wherein said individual is need of IL-2 immunotherapy for treatment of a cancer.

11. The method of claim 10, wherein said individual is also undergoing treatment with an antibody that targets a cell-surface antigen expressed on the surface of cells of said cancer.

12. The method of claim 11, wherein said antibody is an immunoglobulin G1 (IgG1) monoclonal antibody.

13. The method of claim 10, wherein said cancer is a B-cell lymphoma.

14. The method of claim 13, wherein said B-cell lymphoma is non-Hodgkin's B-cell lymphoma.

15. The method of claim 10, wherein said cancer is selected from the group consisting of breast cancer, ovarian cancer, cervical cancer, prostate cancer, colon cancer, melanoma, renal cell carcinoma, acute myeloid leukemia (AML); and chronic lymphocytic leukemia (CLL).

16. The method of claim 9, wherein the allelic pattern for said FcγRIIIA gene is detected by a method selected from the group consisting of allele specific hybridization, primer specific extension, oligonucleotides ligation assay, restriction enzyme site analysis, and single-stranded conformation polymorphism analysis.

17. A method for enhancing immune function of an individual that comprises the homozygous Fc gamma RIIIA (FcγRIIIA) 158F/F genotype, said method comprising administering interleukin-2 immunotherapy to said individual.

18. The method of claim 17, wherein said IL-2 immunotherapy comprises administering at least one therapeutically effective dose of IL-2 or biologically active variant thereof to said individual.

19. The method of claim 18, wherein multiple therapeutically effective doses of IL-2 or variant thereof are administered to said individual.

20. The method of claim 19, wherein said IL-2 or variant thereof is administered according to a daily dosing regimen.

21. The method of claim 19, wherein said IL-2 or variant thereof is administered according to a twice-a-week or three-times-a-week dosing regimen.

22. The method of claim 17, wherein said IL-2 or variant thereof is administered subcutaneously.

23. The method of claim 17, wherein said IL-2 or variant thereof is provided in a pharmaceutical composition selected from the group consisting of a monomeric IL-2 pharmaceutical composition, a multimeric IL-2 composition, a lyophilized IL-2 pharmaceutical composition, and a spray-dried IL-2 pharmaceutical composition.

24. The method of claim 17, wherein said IL-2 is recombinantly produced IL-2 having an amino acid sequence for human IL-2 or a variant thereof having at least 70% sequence identity to the amino acid sequence for human IL-2.

25. The method of claim 24, wherein said variant there of is des-alanyl-1, serine 125 human interleukin-2.

26. The method of claim 17, further comprising administering to said individual an immunoglobulin G1 (IgG1) monoclonal antibody.

27. The method of claim 26, wherein said individual is being treated for a cancer.

28. The method of claim 27, wherein said cancer is a B-cell lymphoma.

29. The method of claim 28, wherein said B-cell lymphoma is non-Hodgkin's B-cell lymphoma.

30. The method of claim 29, wherein said IgG1 monoclonal antibody is an anti-CD20 antibody or antigen-binding fragment thereof.

31. The method of claim 27, wherein said cancer is selected from the group consisting of breast cancer, ovarian cancer, cervical cancer, prostate cancer, colon cancer, melanoma, renal cell carcinoma, acute myeloid leukemia (AML), and chronic lymphocytic leukemia (CLL).

32. The method of claim 27, wherein said IgG1 monoclonal antibody is selected from the group consisting of Therex, MDX-010, EMD 72000, Erbitux, WX-G250, IDM-1, MDX-210, ZAMYL, Campath, and antigen-binding fragments thereof.

33. A method for enhancing immune function of an individual that comprises the heterozygous Fc gamma receptor IIA (FcγRIIA) 131H/R genotype or the homozygous FcγRIIA 131R/R genotype, said method comprising administering interleukin-2 immunotherapy to said individual.

34. The method of claim 33, wherein said IL-2 immunotherapy comprises administering at least one therapeutically effective dose of IL-2 or biologically active variant thereof to said individual.

35. The method of claim 34, wherein multiple therapeutically effective doses of IL-2 or variant thereof are administered to said individual.

36. The method of claim 35, wherein said IL-2 or variant thereof is administered according to a daily dosing regimen.

37. The method of claim 35, wherein said IL-2 or variant thereof is administered according to a twice-a-week or three-times-a-week twice or thrice-weekly dosing regimen.

38. The method of claim 33, wherein said IL-2 or variant thereof is administered subcutaneously.

39. The method of claim 33, wherein said IL-2 or variant thereof is provided in a pharmaceutical composition selected from the group consisting of a monomeric IL-2 pharmaceutical composition, a multimeric IL-2 composition, a lyophilized IL-2 pharmaceutical composition, and a spray-dried IL-2 pharmaceutical composition.

40. The method of claim 33, wherein said IL-2 is recombinantly produced IL-2 having an amino acid sequence for human IL-2 or a variant thereof having at least 70% sequence identity to the amino acid sequence for human IL-2.

41. The method of claim 40, wherein said variant there of is des-alanyl-1, serine 125 human interleukin-2.

42. The method of claim 33, further comprising administering to said individual an immunoglobulin G1 (IgG1) monoclonal antibody.

43. The method of claim 42, wherein said individual is being treated for a cancer.

44. The method of claim 43, wherein said cancer is a B-cell lymphoma.

45. The method of claim 44, wherein said B-cell lymphoma is non-Hodgkin's B-cell lymphoma.

46. The method of claim 45, wherein said IgG1 monoclonal antibody is an anti-CD20 antibody or antigen-binding fragment thereof.

47. The method of claim 43, wherein said cancer is selected from the group consisting of breast cancer, ovarian cancer, cervical cancer, prostate cancer, colon cancer, melanoma, renal cell carcinoma, acute myeloid leukemia (AML), and chronic lymphocytic leukemia (CLL).

48. The method of claim 43, wherein said IgG1 monoclonal antibody is selected from the group consisting of Therex, MDX-010, EMD 72000, Erbitux, WX-G250, IDM-1, MDX-210, ZAMYL, Campath, and antigen-binding fragments thereof.

49. A method for treating a cancer in an individual comprising a homozygous Fc gamma IIIA (FcγRIIIA) 158F/F genotype, said method comprising administering interleukin-2 immunotherapy to said individual.

50. The method of claim 49, wherein said IL-2 immunotherapy comprises administering at least one therapeutically effective dose of IL-2 or biologically active variant thereof to said individual.

51. The method of claim 50, wherein multiple therapeutically effective doses of IL-2 or variant thereof are administered to said individual.

52. The method of claim 51, wherein said IL-2 or variant thereof is administered according to a daily dosing regimen.

53. The method of claim 51, wherein said IL-2 or variant thereof is administered according to a twice-a-week or three-times-a-week twice or thrice-weekly dosing regimen.

54. The method of claim 49, wherein said IL-2 or variant thereof is administered subcutaneously.

55. The method of claim 49, wherein said IL-2 or variant thereof is provided in a pharmaceutical composition selected from the group consisting of a monomeric IL-2 pharmaceutical composition, a multimeric IL-2 composition, a lyophilized IL-2 pharmaceutical composition, and a spray-dried IL-2 pharmaceutical composition.

56. The method of claim 49, wherein said IL-2 is recombinantly produced IL-2 having an amino acid sequence for human IL-2 or a variant thereof having at least 70% sequence identity to the amino acid sequence for human IL-2.

57. The method of claim 56, wherein said variant there of is des-alanyl-1, serine 125 human interleukin-2.

58. The method of claim 49, further comprising administering to said individual an immunoglobulin G1 (IgG1) monoclonal antibody.

59. The method of claim 58, wherein said individual is being treated for a cancer.

60. The method of claim 59, wherein said cancer is a B-cell lymphoma.

61. The method of claim 60, wherein said B-cell lymphoma is non-Hodgkin's B-cell lymphoma.

62. The method of claim 61, wherein said IgG1 monoclonal antibody is an anti-CD20 antibody or antigen-binding fragment thereof.

63. The method of claim 59, wherein said cancer is selected from the group consisting of breast cancer, ovarian cancer, cervical cancer, prostate cancer, colon cancer, melanoma, renal cell carcinoma, acute myeloid leukemia (AML), and chronic lymphocytic leukemia (CLL).

64. The method of claim 59, wherein said IgG1 monoclonal antibody is selected from the group consisting of Therex, MDX-010, EMD 72000, Erbitux; WX-G250, IDM-1, MDX-210, ZAMYL, Campath, and antigen-binding fragments thereof.

65. A method for treating a cancer in an individual comprising a heterozygous Fc gamma IIA (FcγRIIA) 131H/R genotype or a homozygous FcγRIIA 131R/R genotype, said method comprising administering interleukin-2 immunotherapy to said individual.

66. The method of claim 65, wherein said IL-2 immunotherapy comprises administering at least one therapeutically effective dose of IL-2 or biologically active variant thereof to said individual.

67. The method of claim 66, wherein multiple therapeutically effective doses of IL-2 or variant thereof are administered to said individual.

68. The method of claim 67, wherein said IL-2 or variant thereof is administered according to a daily dosing regimen.

69. The method of claim 67, wherein said IL-2 or variant thereof is administered according to a twice-a-week or three-times-a-week twice or thrice-weekly dosing regimen.

70. The method of claim 65, wherein said IL-2 or variant thereof is administered subcutaneously.

71. The method of claim 65, wherein said IL-2 or variant thereof is provided in a pharmaceutical composition selected from the group consisting of a monomeric IL-2 pharmaceutical composition, a multimeric IL-2 composition, a lyophilized IL-2 pharmaceutical composition, and a spray-dried IL-2 pharmaceutical composition.

72. The method of claim 65, wherein said IL-2 is recombinantly produced IL-2 having an amino acid sequence for human IL-2 or a variant thereof having at least 70% sequence identity to the amino acid sequence for human IL-2.

73. The method of claim 72, wherein said variant there of is des-alanyl-1, serine 125 human interleukin-2.

74. The method of claim 65, further comprising administering to said individual an immunoglobulin G1 (IgG1) monoclonal antibody.

75. The method of claim 74, wherein said individual is being treated for a cancer.

76. The method of claim 75, wherein said cancer is a B-cell lymphoma.

77. The method of claim 76, wherein said B-cell lymphoma is non-Hodgkin's B-cell lymphoma.

78. The method of claim 77, wherein said IgG1 monoclonal antibody is an anti-CD20 antibody or antigen-binding fragment thereof.

79. The method of claim 75, wherein said cancer is selected from the group consisting of breast cancer, ovarian cancer, cervical cancer, prostate cancer, colon cancer, melanoma, renal cell carcinoma, acute myeloid leukemia (AML), and chronic lymphocytic leukemia (CLL).

80. The method of claim 75, wherein said IgG1 monoclonal antibody is selected from the group consisting of Therex, MDX-010, EMD 72000, Erbitux, WX-G250, IDM-1, MDX-210, ZAMYL, Carnpath, and antigen-binding fragments thereof.

81. A kit for use in a diagnostic method for predicting therapeutic response to interleukin-2 (IL-2) immunotherapy in an individual in need thereof, said kit comprising at least one probe or primer that specifically hybridizes adjacent to or at a polymorphic region of the Fc gamma receptor IIIA (FcγRIIA) gene, said polymorphic region comprising nucleotides encoding the FcγRIIIA 158F allele.

82. The kit of claim 81, further comprising instructions for use.

83. A kit for use in a diagnostic method for predicting therapeutic response to interleukin-2 (IL-2) immunotherapy in an individual in need thereof, said kit comprising at least one probe or primer that specifically hybridizes adjacent to or at a polymorphic region of the Fc gamma receptor IIA (FcγRIIA) gene, said polymorphic region comprising nucleotides encoding the FcγRIIA 131R allele.

84. The kit of claim 83, further comprising instructions for use.

85. A diagnostic method for predicting therapeutic response to interleukin-2 (IL-2) immunotherapy in an individual in need thereof, said method comprising detecting the allelic pattern for the Fc gamma receptor IIIA (FcγRIIIA) gene of said individual, wherein the presence of the homozygous FcγRIIIA 48L/L genotype, the heterozygous FcγRIIIA 48L/R genotype, or the heterozygous FcγRIIIA 48L/H genotype is indicative of an individual that will exhibit a positive therapeutic response to said IL-2 immunotherapy.

86. The method of claim 85, wherein said individual is need of IL-2 immunotherapy for treatment of a cancer.

87. The method of claim 86, wherein said individual is also undergoing treatment with an antibody that targets a cell-surface antigen expressed on the surface of cells of said cancer.

88. The method of claim 87, wherein said antibody is an immunoglobulin G1 (IgG1) monoclonal antibody.

89. The method of claim 86, wherein said cancer is a B-cell lymphoma.

90. The method of claim 89, wherein said B-cell lymphoma is non-Hodgkin's B-cell lymphoma.

91. The method of claim 86, wherein said cancer is selected from the group consisting of breast cancer, ovarian cancer, cervical cancer, prostate cancer, colon cancer, melanoma, renal cell carcinoma, acute myeloid leukemia (AML); and chronic lymphocytic leukemia (CLL).

92. The method of claim 85, wherein the allelic pattern for said FcγRIIIA gene is detected by a method selected from the group consisting of allele specific hybridization, primer specific extension, oligonucleotides ligation assay, restriction enzyme site analysis, and single-stranded conformation polymorphism analysis.

93. A method for enhancing immune function of an individual that comprises the homozygous Fc gamma RIIIA (FcγRIIIA) 48L/L genotype, said method comprising administering interleukin-2 immunotherapy to said individual.

94. The method of claim 93, wherein said IL-2 immunotherapy comprises administering at least one therapeutically effective dose of IL-2 or biologically active variant thereof to said individual.

95. The method of claim 94, wherein multiple therapeutically effective doses of IL-2 or variant thereof are administered to said individual.

96. The method of claim 95, wherein said IL-2 or variant thereof is administered according to a daily dosing regimen.

97. The method of claim 95, wherein said IL-2 or variant thereof is administered according to a twice-a-week or three-times-a-week dosing regimen.

98. The method of claim 93, wherein said IL-2 or variant thereof is administered subcutaneously.

99. The method of claim 93, wherein said IL-2 or variant thereof is provided in a pharmaceutical composition selected from the group consisting of a monomeric IL-2 pharmaceutical composition, a multimeric IL-2 composition, a lyophilized IL-2 pharmaceutical composition, and a spray-dried IL-2 pharmaceutical composition.

100. The method of claim 93, wherein said IL-2 is recombinantly produced IL-2 having an amino acid sequence for human IL-2 or a variant thereof having at least 70% sequence identity to the amino acid sequence for human IL-2.

101. The method of claim 100, wherein said variant there of is des-alanyl-1, serine 125 human interleukin-2.

102. The method of claim 93, further comprising administering to said individual an immunoglobulin G1 (IgG1) monoclonal antibody.

103. The method of claim 102, wherein said individual is being treated for a cancer.

104. The method of claim 103, wherein said cancer is a B-cell lymphoma.

105. The method of claim 104, wherein said B-cell lymphoma is non-Hodgkin's B-cell lymphoma.

106. The method of claim 105, wherein said IgG1 monoclonal antibody is an anti-CD20 antibody or antigen-binding fragment thereof.

107. The method of claim 103, wherein said cancer is selected from the group consisting of breast cancer, ovarian cancer, cervical cancer, prostate cancer, colon cancer, melanoma, renal cell carcinoma, acute myeloid leukemia (AML), and chronic lymphocytic leukemia (CLL).

108. The method of claim 103, wherein said IgG1 monoclonal antibody is selected from the group consisting of Therex, MDX-010, EMD 72000, Erbitux, WX-G250, IDM-1, MDX-210, ZAMYL, Campath, and antigen-binding fragments thereof.

109. A method for treating a cancer in an individual comprising a heterozygous Fc gamma IIA (FcγRIIA) 131H/R genotype or a homozygous FcγRIIA 131R/R genotype, said method comprising administering interleukin-2 immunotherapy to said individual.

110. The method of claim 109, wherein said IL-2 immunotherapy comprises administering at least one therapeutically effective dose of IL-2 or biologically active variant thereof to said individual.

111. The method of claim 110, wherein multiple therapeutically effective doses of IL-2 or variant thereof are administered to said individual.

112. The method of claim 111, wherein said IL-2 or variant thereof is administered according to a daily dosing regimen.

113. The method of claim 111, wherein said IL-2 or variant thereof is administered according to a twice-a-week or three-times-a-week twice or thrice-weekly dosing regimen.

114. The method of claim 109, wherein said IL-2 or variant thereof is administered subcutaneously.

115. The method of claim 109, wherein said IL-2 or variant thereof is provided in a pharmaceutical composition selected from the group consisting of a monomeric IL-2 pharmaceutical composition, a multimeric IL-2 composition, a lyophilized IL-2 pharmaceutical composition, and a spray-dried IL-2 pharmaceutical composition.

116. The method of claim 109, wherein said IL-2 is recombinantly produced IL-2 having an amino acid sequence for human IL-2 or a variant thereof having at least 70% sequence identity to the amino acid sequence for human IL-2.

117. The method of claim 116, wherein said variant there of is des-alanyl-1, serine 125 human interleukin-2.

118. The method of claim 109, further comprising administering to said individual an immunoglobulin G1 (IgG1) monoclonal antibody.

119. The method of claim 118, wherein said individual is being treated for a cancer.

120. The method of claim 119, wherein said cancer is a B-cell lymphoma.

121. The method of claim 120, wherein said B-cell lymphoma is non-Hodgkin's B-cell lymphoma.

122. The method of claim 121, wherein said IgG1 monoclonal antibody is an anti-CD20 antibody or antigen-binding fragment thereof.

123. The method of claim 119, wherein said cancer is selected from the group consisting of breast cancer, ovarian cancer, cervical cancer, prostate cancer, colon cancer, melanoma, renal cell carcinoma, acute myeloid leukemia (AML), and chronic lymphocytic leukemia (CLL).

124. The method of claim 119, wherein said IgG1 monoclonal antibody is selected from the group consisting of Therex, MDX-010, EMD 72000, Erbitux, WX-G250, IDM-1, MDX-210, ZAMYL, Campath, and antigen-binding fragments thereof.

125. A kit for use in a diagnostic method for predicting therapeutic response to interleukin-2 (IL-2) immunotherapy in an individual in need thereof, said kit comprising at least one probe or primer that specifically hybridizes adjacent to or at a polymorphic region of the Fc gamma receptor IIIA (FcγRIIIA) gene, said polymorphic region comprising nucleotides encoding the FcγRIIIA 48L allele.

126. The kit of claim 125, further comprising instructions for use.
